(19) United States
(12) Patent Application Publication
(10) Pub. No.: US 2025/0131749 A1
(43) Pub. Date: Apr. 24, 2025

(54) SYSTEM AND METHODS FOR ANALYZING MULTICOMPONENT CELL AND MICROBE SOLUTIONS AND METHODS OF DIAGNOSING BACTEREMIA USING THE SAME

(71) Applicant: The Regents of the University of Colorado, a body corporate, Denver, CO (US)

(72) Inventors: Hannah R. Middlestead, Nederland, CO (US); Theodore W. Randolph, Niwot, CO (US); Christopher Calderon, Denver, CO (US)

(21) Appl. No.: 18/701,601

(22) PCT Filed: Oct. 15, 2022
(86) PCT No.: PCT/US22/46801

§ 371 Date: Apr. 15, 2024

Related U.S. Application Data
(60) Provisional application No. 63/256,545, filed on Oct. 16, 2021.

(51) Int. Cl. G06V 20/69; C12M 1/00; C12M 1/34; G06V 10/25; G06V 10/80; G06V 10/82

(52) U.S. Cl. CPC G06V 20/698; C12M 41/36; C12M 47/02; G06V 10/25; G06V 10/80; G06V 10/82; G06V 20/693; G06V 20/695; G06V 2201/03

(57) ABSTRACT

The invention includes systems and methods to combine acoustic sorting, high-throughput imaging technology and machine learning, such as convolutional neural networks (ConvNet) analysis, to analyze cells, pathogens, and other target particles from biological samples resolvable by high-throughput imaging microscopy, or other comparable instrument.

### CROSS-REFERENCE TO RELATED APPLICATIONS

This application claims the benefit of and priority to U.S. Provisional Application No. 63/256,545, filed Oct. 16, 2021. The entire specification and figures of the above-referenced application are hereby incorporated, in their entirety by reference.

### STATEMENT OF FEDERALLY SPONSORED RESEARCH

This invention was made with government support under grant number R43EB029863 awarded by the National Institutes of Health. The government has certain rights in the invention.

### TECHNICAL FIELD

The invention includes novel systems, methods and compositions for the isolation, detection, and quantification of one or more microparticles, such as bacteria in a biological sample, which may further be accomplished in real-time. The invention further includes novel systems, methods and compositions for the isolation, detection, and quantification of one or more microparticles, such as bacteria in a biological sample based on a physiological characteristic of the microparticles, such as antibiotic resistance, such as transfection and transduction, which may further be accomplished in real-time. The invention includes novel systems, methods and compositions for the analysis of transduction rates of T cells during the culture of T cells to form CAR-T cells used in cell therapy for the treatment of cancer using immunotherapy, which may further be accomplished in real-time.

### BACKGROUND

High-throughput analysis of microscopy images has numerous potential applications in the field of healthcare. One example is the analysis of cells and microparticles present biological fluid samples, such a blood or urine. In this application, the timely diagnosis of pathogenic cells, such as bacteria, and in particular multi-drug resistant bacteria is hindered by the low throughput of conventional microscopy and other cell identification techniques, and the long times required for these analyses. Even when automated microscope slide readers are employed, the throughput is limited by sample preparation time, the need to apply time-consuming staining techniques, the small volume of sample that can be analyzed per microscope slide, and the challenges of detecting and identifying minute levels of foreign infectious microorganisms within the vast numbers of normal cells found in typical biological samples. In order to detect and identify small populations of foreign infectious microorganisms, biological samples must typically be cultured to allow the number of foreign infectious microorganisms to increase to more readily detectable levels, a process that can require multiple days of culturing and further limit throughput. Moreover, culturing procedures, such as the detection of bacteremia through blood culturing techniques are susceptible to Type II errors. Thus, identification of pathogens within biological samples often takes days and involves complicated procedures, a situation that may unduly delay effective treatment such as the appropriate selection of an antibiotic. In some instances, these delays have proved to be fatal to patients or have caused unnecessary suffering. A common practice in treating infected patients is the use of broad-spectrum antibiotics. However, due to the problem of bacterial resistance to many antibiotics, broad-spectrum antibiotics may not effectively treat many infections.

Further, for same patient populations such as premature neonates, analysis of bacteremia by blood culture requires ˜1 mL of blood to be moderately accurate, which is often not possible with neonates, especially with the majority of neonatal sepsis cases being in infants with low birth weights. Moreover, side-effects from inappropriately applied or unnecessary antibiotics may put these premature neonates at risk for severe complications, such as kidney failure, and damage to the emerging gut microbiome. Indeed, neonatal sepsis is a leading cause of infant death throughout the world. Insufficient diagnostic testing is a main contributing factor, with the current diagnostic method being blood culture. Many cases of infectious disease can be prevented or more effectively and promptly treated if rapid and accurate diagnosis is available. Thus, there is a need for rapid and accurate methods for identifying infectious pathogens within biological samples.

Attempts have been made to addresses these concerns but have fallen short for a number of technical reasons. For example, Smith et al., (U.S. Pat. No. 10,255,693) describes a method for detecting and classifying particles found on traditional microscopy slides collected using a low number of repeat magnifications on a single slide. While Smith does implement some neural network-based applications, the system is designed for analyzing a small number of images characterizing a single slide and requires a priori knowledge of the type of objects of interest. Smith also requires detailed label annotation instead of flow microscopy settings not requiring the detailed label annotation of each image, thus limiting its throughput, effectiveness and commercial applicability. In another example, Krause et al., (U.S. Pat. No. 10,303,979) describes a Convolutional Neural Network-based analysis for analyzing microscopy images in order to identify the contents of the slide as well as to segment the images into individual cells and cell types. Again, this application does not allow for real-time imagining and analysis of flow microscopy nor does it allow one to statistically verify confidence in known particles or identify faults or novel observations (those classes not in the training data) in the test data. In another example, Grier et al., (U.S. Pat. No. 10,222,315) describe the application of holographic microscopy techniques for characterizing protein aggregates. However, this application requires the precise calibration of various lasers applied to a biological sample and the concurrent measurement of their diffraction patterns. As a result, this system is less adaptable to various applications and must be precisely maintained diminishing its commercial effectiveness.

As described below, the present inventors demonstrate a rapid and accurate machine learning-based system to analyse digital microscopy images of cells found in as little as 50 μL of a biological fluid, which can identify any bacterial or fungal pathogenic infection within a 1-hr total analysis timeframe. The technique can also be utilized to characterize changes in engineered cell lines.

### SUMMARY OF THE INVENTION

One aspect of the current inventive technology includes systems and methods that may combine high-throughput flow or static imaging technology and machine learning, such as convolutional neural networks, in variety of medical applications. In certain embodiments, the approaches described herein may use high-throughput flow imaging microscopy instrumentation and machine learning module application, such as a digital filter, which may include a computer executable program, hardware application or combination of the same, that can differentiate different microparticles by one or more characteristic. In one embodiment, a digital filter can be a Convolutional neural network (ConvNet) that can analysis, to analyze cells, pathogens, and other target particles resolvable by high-throughput flow imaging microscopy, or other comparable instrument.

In one aspect, the invention includes novel systems, methods and compositions for the separation and identification of microparticles, such as bacterium, also referred to herein as microbes, in a biological sample. In one embodiment, a biological sample from a subject may undergo acoustic separation followed by flow imaging microscopy with parameters that have been adjusted so as to obtain multiple images of a microparticle or feature of interest, and ending with machine learning analysis. This process allows microbes to be isolated within an infected biological sample, and subsequently be identified by species. This process is a much more informative, accurate, and expedited form of diagnostic techniques currently available. This can be attributed to the methodical processing of the sample, and the high accuracy of species determination achieved by the unique machine learning-based automated classifier.

In another specific application, a biological sample, and preferably a blood sample is processed in a three-step sequence: acoustic separation by a separation module, high-resolution oil-immersion flow microscopy utilizing a digital image capture module, and classification by the convolutional neural network. Acoustic separation removes larger blood cells from blood samples, leaving smaller microbial cells. The sample is passed through a microfluidic imaging device, and multiple images of each cell in the sample are recorded. The images captured are processed by a convolutional neural network incorporating a cross-entropy technique and a moving sliding window of classification scores to make use of image redundancy. The convolutional neural network first classifies images of each cell as being images of either microbes or blood cells. Every image that the network identified as an image of a microbe is then fed into a second neural network that identifies the species of the microbe. Each individual image receives a classification likelihood, for each class. To increase the accuracy of the system and reduce the instances or chance of a false positive, image redundancy techniques are used where multiple, sequential images of are recorded during passage of cells through the flow imaging microscope. Using these sequentially recorded images, the accuracy of identifying a microbe within a given time series of images can be increased by taking into account (e.g., using a sliding window calculation) the likely identity of the images that appear in the time series before and after the image of interest.

In another specific application, a biological sample, such as a urine sample is processed in a three-step sequence: acoustic separation by a separation module, high-resolution oil-immersion flow microscopy utilizing a digital image capture module, and classification by the convolutional neural network, generally referred to herein as a machine learning module. Acoustic separation removes larger particles and subject-derived cells from the urine samples, leaving smaller microbial cells. The sample is passed through a microfluidic imaging device, and multiple images of each cell in the sample are recorded. The images captured are processed by a convolutional neural network incorporating a cross-entropy technique and a moving mean calculation of classification scores to make use of image redundancy. The convolutional neural network first classifies images of each cell as being images of either microbes or human-derived cells. Every image that the network identifies as an image of a microbe is then fed into a second neural network that identifies the species of the microbe. Each individual image receives a classification likelihood, for each class. The system of the invention can further identify, and group microbes based on one or more drug-resistance characteristics. In this embodiment, training data sets may be grouped by drug resistances, in addition to the typical grouping of subspecies or species. as described herein. To increase the accuracy of the system and reduce the instances or chance of a false positive, image redundancy techniques are used where multiple images of each cell or microbe are recorded in a correlated time series as the sample flows through the flow microscopy instrument, and the likely identity (as determined by the machine learning module) of the image before and the image after an image of interest are also taken into account when determining the identity of a cell or microbe in the image of interest using a moving mean calculation or other weighted average technique.

In another specific application, a biological sample, such as a spinal fluid sample or a sputum fluid sample is processed in a three-step sequence: acoustic separation by a separation module, high-resolution oil-immersion flow microscopy utilizing a digital image capture module, and classification by the convolutional neural network, generally referred to herein as a machine learning module. Acoustic separation removes larger particles and cells from the biological samples, leaving smaller microbial cells. The sample is passed through a microfluidic imaging device, and multiple digital images of each cell in the sample are recorded. The images captured are processed by a convolutional neural network incorporating a cross-entropy technique and a moving mean calculation of classification scores to make use of image redundancy. The convolutional neural network first identifies whether each cell image is an image of either a microbe or a non-microbial particle present in the sputum or spinal fluid sample. Every image that is identified as an image of a microbe is then processed using a second neural network that identifies the pathogen species. Each individual image is assigned a classification likelihood, for each class. The system of the invention can further identify, and group microbes based on one or more drug-resistance characteristics. In this embodiment, training data sets may be grouped by drug resistances, in addition to the typical grouping of subspecies or species. as described herein. Each individual image receives a classification likelihood, for each class. To increase the accuracy of the system and reduce the instances or chance of a false positive, image redundancy techniques are used where multiple images of each cell or microbe are recorded in a correlated time series as the sample flows through the flow microscopy instrument, and the likely identity (as determined by the machine learning module) of the image before and the image after an image of interest are also taken into account when determining the identity of a cell or microbe in the image of interest using a moving mean calculation or other weighted average technique.

In another specific application, invention includes the analysis of transduction rates in cultured T-cells during the production of CAR-T cells used in cell therapy for the treatment of cancer using immunotherapy. In this aspect of the invention, transduction rate determinations may be made by establishing training data sets that are grouped by transduced samples, and non-transduced samples, at various time points. For unsupervised learning, transduced and/or non-transduced cells at various time points can be used to estimate low-dimensional representations of the images of interest via feature techniques such as variational auto-encoders (VAEs) and generative adversarial networks (GANs). Unsupervised representations can be visualized for exploratory data analysis (EDA) or quality control (QC) applications using additional post-processing steps such as t-distributed stochastic neighbor embedding (t-SNE) or Uniform Manifold Approximation and Projection (UMAP) techniques. The acoustic separation of microparticles removing cell debris and other small particles can assist both supervised and unsupervised training. Physical sorting can be combined with computational filtering to better focus on cells or cell components of interest. In addition, the image redundancy technique described previously can help in capturing multiple views of cells of interest in both supervised and unsupervised training and evaluation phases.

In additional embodiment, the invention may be used to make diagnostics and therapeutic treatment decisions. For example, using the information obtained from the inventive system, the present inventors can determine the concentration of bacteria within a biological sample, such as blood, urine, sputum and subsequently determine the severity of infection. Further, using the information obtained from the inventive system, the present inventors can determine the species of the microbial infection, and drug resistance characteristics of microbes within a biological sample. These determinations allow appropriate measures to be taken regarding patient care, and the rigor of treatment needed.

In another aspect, the invention may include one or more of the following preferred embodiments:

1. A system for analyzing a biological sample comprising:
a biological sample containing a quantity of microparticles;
an image capture module configured to capture a plurality of digital image signals of said microparticles present in said biological sample;
a machine learning module configured to process the digital image signals from said image capture module further comprising:
   a digital filter to differentiate the images of microparticles of interest from images of microparticles in said biological sample; and
   a convolutional neural network configured to further identify said microparticles of interest.

2. The system of embodiment 1, further comprising a separation module configured to separate the microparticles in said biological sample into a collection outlet stream containing predominantly microparticles of interest, and a waste stream containing predominately other particles found in said biological sample.

3. The system of embodiment 2, wherein said image capture module is further configured to capture a plurality of digital image signals of said microparticles present in said collection outlet of said biological sample.

4. The system of any of embodiments 1 and 3, wherein said digital filter is further configured to differentiate images of microparticles of interest from images of the microparticles in said collection outlet of said biological sample.

5. The system of embodiment 1, wherein said plurality of digital image signals comprises a plurality of digital image signals captured sequentially.

6. The system of embodiment 1, wherein said plurality of digital image signals comprises a plurality of digital image signals captured non-sequentially.

7. The system of any of embodiments 1 to 6, wherein digital images of microparticles are obtained after adhering the microparticles to a membrane.

8. The system of any of embodiments 1 to 6, wherein said biological sample comprises a static or flowing liquid suspension.

9. The system of any of embodiment 1 to 8, wherein said wherein the biological sample comprises a biological sample selected from the group consisting of: sputum, oral fluid, amniotic fluid, blood, a blood fraction, bone marrow, a biopsy samples, urine, semen, stool, vaginal fluid, peritoneal fluid, pleural fluid, tissue explant, mucous, lymph fluid, organ culture, cell culture, or a fraction or derivative thereof or isolated therefrom

10. The method of embodiment 9, wherein said blood sample comprises a blood sample having a volume of about 25 to 100 microliters.

11. The system of embodiment 1, wherein said microparticles in said biological sample comprise microbial and non-microbial microparticles.

12. The system of embodiment 11, wherein said non-microbial microparticles comprise cells.

13. The system of embodiment 1, and wherein said microbial microparticles comprises pathogenic microbes.

14. The system of embodiment 1, wherein said separation module comprises an acoustic separation module.

15. The system of embodiment 14, wherein said acoustic separation module separates said microparticles according to size, and/or compressibility.

16. The system of embodiment 1, wherein said image capture module comprises a high-throughput imaging instrument capable of imaging flowing or static suspensions of microparticles.

17. The system of embodiment 16, wherein said high-throughput imaging instrument comprises a high-throughput microfluidic imaging instrument capable of imaging flowing or static liquid suspensions.

18. The system of embodiment 17, wherein said high-throughput microfluidic imaging instrument is selected from the group consisting of: a high-throughput flow imaging microscopy (FIM) instrument, high-throughput imaging microscopy instrument; a high-resolution oil-immersion flow microscopy; a high-resolution oil-immersion microscopy.

19. The system of embodiment 16, wherein said high-throughput imaging instrument captures multiple, sequential digital image signals of said microparticles using high-resolution oil-immersion microscopy.

20. The system of embodiment 16, wherein said high-throughput imaging instrument captures multiple, non-sequential digital image signals of said microparticles using high-resolution oil-immersion microscopy.

21. The system of embodiment 1, wherein said digital filter comprises a convolutional neural network further comprising a machine learning-based automated classifier configured to determine if the microparticles are a microbe of interest, or a subject-derived cell, and/or wherein said digital filter comprises a convolutional neural network further comprising a machine learning-based embedding scheme configured to determine if the cell culture components comprising the microparticles are microbes of interest, or a subject-derived cells.

22. The system of embodiment 21, wherein said convolutional neural network comprises a machine learning-based automated classifier configured to identify the microbe of interest by genus, species, phenotypic characteristic, genotypic characteristic, or one or more antibiotic resistance characteristics.

23. The system of embodiment 1, wherein said system generates a reference dataset by passing a reference sample comprising a biological sample through said system.

24. The system of embodiment 1, wherein said system generates a test dataset by passing a test sample comprising a biological sample through said system, which can be compared to said reference dataset.

25. The system of embodiment 1, and wherein said microparticle of interest is correlated with a disease condition.

26. The system of embodiment 25, wherein said disease condition comprises sepsis.

27. The system of embodiment 1, wherein said digital image signals of said microparticles comprise digital image signals selected from the group consisting of: brightfield images, darkfield images, fluorescent images, infrared spectroscopic images, and Raman spectroscopic images.

28. The system of embodiment 1, wherein said machine learning module further comprises a fusion module adapted to combine signal modalities.

29. The system of embodiment 28, wherein said fusion module is adapted to fuse embeddings in signals from two or more modalities.

30. The system of embodiment 29, wherein said modalities comprise brightfield microscopy, darkfield microscopy, fluorescence spectroscopy, a Raman spectroscopy, infrared spectroscopy or other orthogonal particle characterization methods.

31. The system of embodiment 1, wherein said machine learning module configured to process the digital image signals from said image capture module comprises a machine learning module configured to extract one or more features of said microparticles of interest by machine learning including supervised learning, or unsupervised learning.

32. The system of embodiment 1, wherein said biological sample comprises a pharmaceutical sample.

33. The system of embodiment 1, wherein said pharmaceutical sample is selected from the group consisting of: biopharmaceutical suspensions, biopharmaceutical formulations, protein biologic formulations, protein biologic suspensions, antibody formulations, antibody suspensions, antibody-drug conjugates formulations, antibody-drug conjugates suspensions, fusion protein formulations, fusion protein suspensions, vaccine formulations, and vaccine suspensions.

34. A system for characterizing changes in cell populations:
a biological sample containing a quantity of a cell culture further containing a quantity of engineered cells;
an image capture module configured to capture a plurality of digital image signals of the engineered cells present in said biological sample;
a machine learning module configured to process the digital image signals from said image capture module further comprising a convolutional neural network configured to extract a feature of interest from said images;
one or more additional biological samples containing a quantity of a cell culture containing a quantity of engineered cells applied to the system above and compared to said preceding biological sample, wherein said comparison identifies a characteristic change in said engineered cells between the samples.

35. The system of embodiment 34, further comprising a separation module configured to separate said engineered cells in said biological sample into a collection outlet of said biological sample.

36. The system of embodiments 34 or 35, wherein said plurality of digital image signals comprises a plurality of digital image signals captured sequentially.

37. The system of embodiments 34 or 35, wherein said plurality of digital image signals comprises a plurality of digital image signals captured non-sequentially.

38. The system of any of embodiments 34 to 37, wherein said biological sample comprises a biological sample adhered to a membrane.

39. The system of any of embodiments 34 to 37, wherein said biological sample comprises a static or flowing liquid suspension.

40. The system of embodiment 34, wherein said the biological sample comprises a cell culture containing a quantity of transduced cells, and/or non-transduced cells.

41. The system of embodiment 34, wherein said feature of interest comprises a feature of interest associated with transduced cells, or non-transduced cells.

42. The system of embodiment 41, wherein said transduced cells comprise T cells transduced to form CAR-T cells.

43. The system of embodiment 34, wherein said separation module comprises an acoustic separation module.

44. The system of embodiment 43, wherein said acoustic separation module is configured to separate the cell culture components according to size and/or compressibility.

45. The system of embodiment 34, wherein said image capture module comprises a high-throughput imaging instrument capable of imaging static or flowing liquid suspensions, or microparticles extracted from the liquid suspensions.

46. The system of any of embodiment 44 or 45, wherein said high-throughput microfluidic imaging instrument is selected from the group consisting of: high-throughput microfluidic imaging instrument; a high-throughput flow imaging microscopy (FIM) instrument, high-throughput imaging microscopy instrument; a high-resolution oil-immersion flow microscopy; a high-resolution oil-immersion microscopy.

47. The system of embodiment 45, wherein said high-throughput imaging instrument is configured to capture multiple, sequential digital images of cell culture components using high-resolution oil-immersion microscopy.

48. The system of embodiment 45, wherein said high-throughput imaging instrument captures multiple, non-sequential digital image signals cell culture components using high-resolution oil-immersion microscopy.

49. The system of embodiment 34, wherein said digital filter comprises a machine learning-based automated classifier configured to determine if the cell culture components comprise T cells transduced to form CAR-T cells, or non-transduced T-cells.

50. The system of embodiment 34, wherein said digital filter comprises a machine learning-based embedding scheme configured to determine if the cell culture components comprise transduced CAR-T cells, or non-transduced T-cell.

51. The system of embodiment 34, wherein said system generates a reference dataset by passing a reference sample comprising a biological sample through said system.

52. The system of embodiment 34, wherein said system generates a test dataset by passing a test sample comprising a biological sample through said system, which can be compared to said reference dataset.

53. The system of embodiment 34, and wherein said characteristic change in said engineered cells between the samples is correlated with a disease condition.

54. The system of embodiment 34, wherein said digital image signals comprise digital image signals selected from the group consisting of: brightfield microscopy images, darkfield microscopy images, fluorescence spectroscopy images, infrared spectroscopy images, Raman spectroscopy images, or other orthogonal particle characterization methods.

55. The system of embodiment 34, wherein said machine learning module further comprises a fusion module adapted to combine signal modalities.

56. The system of embodiments 55, wherein said fusion module is adapted to fuse embeddings in signals from two or more modalities.

57. The system of embodiment 56, wherein said modalities comprise brightfield microscopy, darkfield microscopy, fluorescence spectroscopy, infrared spectroscopy, Raman spectroscopy or other orthogonal particle characterization methods.

58. The system of embodiment 34, wherein said machine learning module configured to process the digital image signals from said image capture module comprises a machine learning module configured to extract one or more features of interest by supervised learning or unsupervised learning.

59. A system for characterizing changes in pharmaceutical sample populations:
a pharmaceutical sample containing a quantity of microparticles;
an image capture module configured to capture a plurality of digital image signals of the microparticles present in said pharmaceutical sample;
a machine learning module configured to process the digital image signals from said image capture module further comprising a convolutional neural network configured to extract a feature of interest from said images; and
one or more additional pharmaceutical samples containing a quantity of microparticles applied to the system above and compared to said preceding pharmaceutical sample, wherein said comparison identifies a characteristic change in between the samples.

60. The system of embodiment 59, wherein said pharmaceutical sample is selected from the group consisting of: biopharmaceutical suspensions, biopharmaceutical formulations, protein biologic formulations, protein biologic suspensions, antibody formulations, antibody suspensions, antibody-drug conjugates formulations, antibody-drug conjugates suspensions, fusion protein formulations, fusion protein suspensions, vaccine formulations, and vaccine suspensions.

61. The system of embodiments 59, wherein said plurality of digital image signals comprises a plurality of digital image signals captured sequentially.

62. The system of embodiments 59, wherein said plurality of digital image signals comprises a plurality of digital image signals captured non-sequentially.

63. The system of any of embodiments 59 to 60, wherein said pharmaceutical sample comprises a pharmaceutical sample adhered to a membrane.

64. The system of any of embodiments 59 to 60, wherein said pharmaceutical sample comprises a static or flowing liquid suspension.

65. The system of embodiment 59, further comprising a separation module configured to separate said microparticles in said pharmaceutical sample into a waste outlet and a collection outlet of said pharmaceutical sample.

66. The system of embodiment 65, wherein said separation module comprises an acoustic separation module.

67. The system of embodiment 66, wherein said acoustic separation module separates said microparticles according to size, and/or compressibility.

68. The system of embodiment 59, wherein said image capture module comprises a high-throughput imaging instrument capable of imaging static or dynamic microparticles.

69. The system of embodiment 68, wherein said high-throughput imaging instrument comprises a high-throughput microfluidic imaging instrument capable of imaging static or flowing liquid suspensions.

70. The system of embodiment 69, wherein said high-throughput microfluidic imaging instrument is selected from the group consisting of: a high-throughput flow imaging microscopy (FIM) instrument, high-throughput imaging microscopy instrument; a high-resolution oil-immersion flow microscopy; a high-resolution oil-immersion microscopy.

71. The system of embodiment 71, wherein said high-throughput imaging instrument captures multiple, sequential digital image signals of said microparticles using high-resolution oil-immersion microscopy.

72. The system of embodiment 71, wherein said high-throughput imaging instrument captures multiple, non-sequential digital image signals of said microparticles using high-resolution oil-immersion microscopy.

73. The system of embodiment 59, wherein said system generates a reference dataset by passing a reference sample comprising a pharmaceutical sample through said system.

74. The system of embodiment 59, wherein said system generates a test dataset by passing a test sample comprising a pharmaceutical sample through said system, which can be compared to said reference dataset.

75. The system of embodiment 59, wherein said digital image signals of said microparticles comprise digital image signals selected from the group consisting of: brightfield microscopy, darkfield microscopy, fluorescence spectroscopy, infrared spectroscopy, Raman spectroscopy or other orthogonal particle characterization methods.

76. The system of embodiment 59, wherein said machine learning module further comprises a fusion module adapted to combine signal modalities.

77. The system of embodiment 76, wherein said fusion module is adapted to fuse embeddings in signals from two or more modalities.

78. The system of embodiment 77, wherein said modalities comprise brightfield microscopy, darkfield microscopy, fluorescence spectroscopy, infrared spectroscopy, Raman spectroscopy or other orthogonal particle characterization methods.

79. the system of embodiment 59, wherein said machine learning module configured to process the digital image signals from said image capture module comprises a machine learning module configured to extract one or more features of said microparticles of interest by supervised learning or unsupervised learning.

Other features, objects, and advantages of the invention will be apparent from the Detailed Description, the Figures, the Examples, and the Claims.

### BRIEF DESCRIPTION OF THE FIGURES

Aspects, features, and advantages of the present disclosure will be better understood from the following detailed descriptions taken in conjunction with the accompanying figures, all of which are given by way of illustration only, and are not limiting the presently disclosed embodiments, in which:

FIG. 1: Method of Analysis Pathway. The diagnostic process takes place in three major steps. Initially the separation module is used to remove the larger host cells, in order to purify the sample and make analysis possible. After the microbe-rich sample is procured it is imaged using the an image capture module, and the images are then processed using a specially designed machine learning algorithm.

FIG. 2: Representative Image Capture Module Outputs. Two representative collages are pictured, the left collage using 3 μm beads suspended in PBS buffer, and the right collage using a sample of E. coli cells. The image redundancy is clearly visible in both cases, with the redundancy of images falling between three and seven instances. Exemplary image redundancy effect is highlighted using an orange box to surround a specific repetition.

FIG. 3: Machine Learning Module Classification Pathway. Demonstration of the machine learning module algorithm application. In one embodiment, the microbe rich sample is obtained from the separation module, and then imaged by the image capture module and processed through a machine learning module which uses the workflow shown herein. The digital images are processed by an initial classifying algorithm which determines if each image is a microbe or a host cell, using a sliding window average of width ‘n’ images, where ‘n’ is based on the expected number of redundant image instances. All images identified as microbes are then processed through a second classifier that determines species, using the same parameter ‘n’.

FIG. 4: Separation of Blood and General Microbe Groups. The confusion matrix results of the first step of the machine learning module algorithm are shown. The horizontal axis contains the classes on which the algorithm was trained, and the vertical axis contains the samples that were tested after the determination of parameters by the algorithm training. The correctly classified results are shown in bold font. In step one the microbes are differentiated from host cells, which in this case are blood cells. In the second table shown, out of sample (OoS) test are shown, which allow us to see how a group of images which the classifier was not trained on would perform. In this case and all future cases, “Blood” and “Human Platelets” are used interchangeably. Blood or platelets obtained from another species will be specified.

FIG. 5: Identification of Microbe Species. The confusion matrix results of the second step of the machine learning algorithm are shown above. The horizontal axis contains the classes the algorithm was trained on, and the vertical axis contains the samples that were tested after the determination of parameters by the algorithm training. The correctly classified results are shown in bold font. In step two the microbe species are identified. In the second table shown, out of sample (OoS) test are shown, which allow us to see how a group of images which the classifier was not trained on would perform. In this case and all future cases, “Blood” and “Human Platelets” are used interchangeably. Blood or platelets obtained from another species will be specified.

FIG. 6: Identification of Microbe Species in Murine and Human Blood Samples. In step one of the classification algorithm, the images within the infected sample that are identified as images of microbes are separated from the images identified as blood. The images of microbes images are then processed in the second step of the classifier, where the species of the microbe is determined. In this analysis microbes at a concentration of 100000 CFU/mL were added to Murine blood samples at a total dilution of 10×. The Human blood samples were used to analyze additions of microbes at 10000 CFU/mL and 1000 CFU/mL. Both cases were diluted by 30× in PBS. Additionally, for the table containing human samples a column was added to show the difference between the result of the test, and the result if there were no microbes within the solution (The out of sample blood). This allows us to clarify the results and see that the correct classification is occurring.

FIG. 7: Identification of Microbe Species in Urine Samples. In this application, the two-step method is not needed to process urine samples due the low number of host cells in the solution. Therefore, images were analyzed immediately for species determination. In this analysis microbes at a concentration of 10000 CFU/mL were added to artificial urine samples and then imaged.

FIG. 8: Determination of Drug Susceptibility in Multi-Drug Resistant E. coli. The confusion matrix for a parallel classifier is show, in which each susceptibility was trained on individually, and then the results complied. This allows for a 0-100% identification of susceptibility for each drug, which is necessary for the multi drug resistant microbes used in this case. In the confusion matrice, the horizontal axis contains the classes the algorithm was trained on, individually, and the vertical axis contains the samples that were tested after the determination of parameters by the algorithm training. The incorrectly classified results are shown in bold font, which are only two of twenty-eight cases. The number in parentheses next to each testing case represents the number of independent samples in that specific group.

FIG. 9: Determination of Transduction Rates in CAR-T Cell Therapy using a Classification Model. The confusion matrix results of the machine learning classifier are shown. The horizontal axis contains the classes the algorithm was trained on, and the vertical axis contains the samples that were tested after the determination of parameters by the algorithm training. The training sets for the ‘Transduced’ groups included samples from multiple time points (2) hours, and 164 hours), and the training sets for the ‘Non-transduced’ groups included samples from the same time points for comparison. The correctly classified results are shown in bold font.

FIG. 10: Determination of Transduction Rates in Transduced B. subtilis using a Classification Model. The confusion matrix results of the machine learning classifier are shown. The horizontal axis contains the classes the algorithm was trained on, and the vertical axis contains the samples that were tested after the determination of parameters by the algorithm training. The training sets for the ‘Transduced’ groups included samples from multiple procedures (3 instances) and the training sets for the ‘Non-transduced’ groups included samples from the same procedures for comparison. The correctly classified results are shown in bold font. In this case one of the out of sample tests did not identify correctly, but the other 3 cases did. The out of sample tests were not used for training.

FIG. 11: Illustration of Using Unsupervised Embedding Representations to Characterize CAR-T cell Morphologies Encoded in Images Captured Dynamically with FlowCAM Nano. In the top row, a two-dimensional embedding of a variational autoencoder's representation of images collected from two different cell conditions (embeddings obtained with Uniform Manifold Approximation and Projection for Dimension Reduction); the autoencoder was trained/calibrated using color brightfield image information to obtain its latent space representation. The left columns correspond to transduced cells and the right columns correspond to untreated CAR-T cells; the arrows show representative source FlowCAM Nano images nearest the embedding points where the arrows originate from. The morphology differences between the two distinct cell populations allow discrimination of two cell conditions via neural networks and also enables monitoring the morphology evolution over time. The approach can also be used to detect the presence of new particle populations appearing in the cell imaged in liquid.

FIG. 12: Illustration of Using Unsupervised Embedding Representations to Characterize Protein Aggregate Morphologies Encoded in Images Obtained by Backgrounded Membrane Imaging (BMI). In the top row, a two-dimensional embedding of a variational autoencoder's representation of particle images from two protein conditions are shown (embeddings obtained with Uniform Manifold Approximation and Projection for Dimension Reduction); the autoencoder was trained/calibrated using information from two different modalities to obtain its latent space representation (two separate channels of brightfield and darkfield were fused at the neural network input). The left columns correspond to an unstressed antibody formulation and right columns correspond to stressed antibody formulation; the arrows show representative brightfield BMI images nearest the embedding points where the arrows originate from. The morphology differences between the two distinct particles obtained under different conditions allow discrimination of two conditions via neural networks and the approach also enables monitoring the morphology evolution of the particles over time. The approach can also be used to detect the presence of new particle populations appearing in the particles extracted from the liquid solution via BMI.

### DETAILED DESCRIPTION OF THE INVENTION

In one aspect of the current invention, the present inventors combined high-throughput microfluidic imaging with ConvNets to analyze particles, such as bacterial pathogens in blood, urine and other biological fluid samples among others. High-throughput microfluidic imaging may incorporate microfluidics and light microscopy techniques to capture images of particles larger than approximately 200 nm in a sample. ConvNets are a family of neural networks capable of learning relevant properties of an input image that are useful when performing computer vision tasks such as object identification, classification, and statistical representation. Although the images obtained from the instrument contain a large amount of morphological information about the particles in a sample, it is difficult to manually extract this information from the raw images and to use that information to analyze the particles in a sample. In the present invention, it has been discovered that ConvNets can be trained using high-throughput microfluidic images, where each image is not provided a detailed class label, and the resulting network can be applied in order to extract and utilize the morphological information contained within the image.

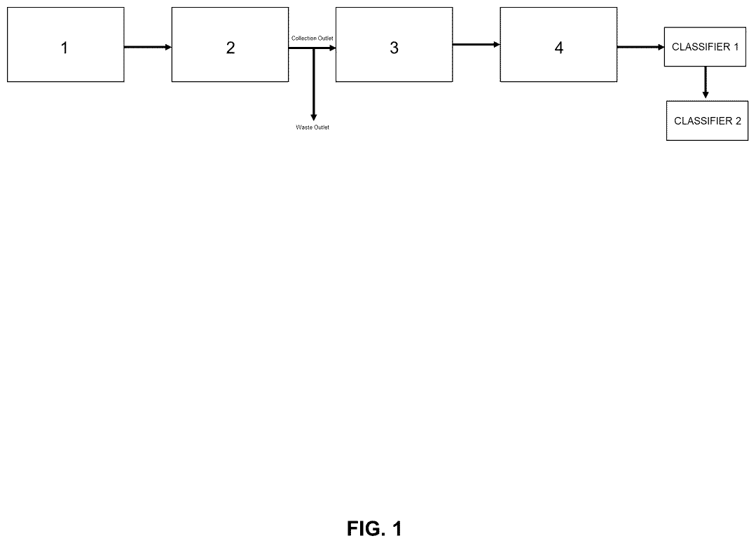

As generally outlined in FIG. 1, further aspects of the inventive technology include systems and methods of applying machine learning to detect and analyze particles, such as bacteria in liquid suspensions in high-throughput microparticle imaging systems. “High-throughput imaging” systems may image particles dynamically in solution (e.g., as done by a FlowCam Nano™ instrument) or particles in a static solution; the imaging system can also refer to techniques where liquid containing particles are transferred to a membrane, the liquid is filtered, and the particles remaining are imaged (e.g., as done by the Aura™ backgrounded membrane imaging system), which can also be referred to generally as static imaging. In one preferred embodiment, a neural network, such as a multi-layer ConvNet, may be trained via an initial training dataset. In this embodiment, at least one reference dataset may be generated by passing a reference sample comprising particles in a liquid suspension, through an image capture module (3), which preferably includes a high-throughput flow microfluidic imaging instrument. In preferred embodiment, the sample may be processed prior to imaging through a separation module (2), and preferably a separation module (2) configured to allow the acoustic separation of microparticles, such as bacteria from a biological sample (1).

Digital images of the particles passing through the device may be captured for later processing. These images may be transmitted to one or more processors, or other similar data processing device or system, where features of interest that may be indicative of bacteria, such as size or shape are extracted. This extraction may be accomplished in a preferred embodiment by a machine learning system, and more preferably a ConvNet Machine learning Module (4) as generally described herein. In a preferred embodiment, at least 10⁴ to 10⁷ or more images of the individual components passing through a high-throughput imaging instrument (also referred to herein as a HTI), may be captured for further extraction and analysis.

Another aspect of the inventive technology may include methods of applying machine learning to detect and analyze cells and microbial pathogens in biological samples in high-throughput systems. In this embodiment, at least one reference dataset may be generated by passing a reference sample, which may preferably comprise cells in a biological sample, such as preferably a blood or urine sample, or more preferably blood or urine sample having a volume of at least 25 to 100 microliters or more, through a HTI or other similar instrument. Exemplary biological samples may include: sputum, oral fluid, amniotic fluid, blood, a blood fraction, bone marrow, a biopsy samples, urine, semen, stool, vaginal fluid, peritoneal fluid, pleural fluid, tissue explant, mucous, lymph fluid, organ culture, cell culture, or a fraction or derivative thereof or isolated therefrom.

Digital images of the individual components of the biological sample passing through the HTI may be captured for later processing. These images may be transmitted to one or more processors, or other similar data processing device or system, where features of interest are extracted. In one preferred embodiment, an extracted feature of interest is correlated with a known disease condition, such as sepsis. In alternative embodiments, a disease condition may be associated with the type or quantity of the extracted feature of interest or the type and quantity of cells found in the biological sample. This extraction may be accomplished, in a preferred embodiment, by a machine learning system, and more preferably a ConvNet Feature Extraction Module. In another preferred embodiment, at least 10⁴ to 10⁷ images of the individual components passing through said HTI instrument may be captured for further extraction and analysis.

In one optional embodiment, one or more additional reference datasets may be generated by the process generally described above. In this optional embodiment, one, or a plurality of additional samples comprising liquid suspensions of cells resulting from infection, or contamination, or a disease state may be processed by a separation module (2) and then allowed to pass through, for example, a HTI instrument. Digital images of the individual components of each sample may be captured and further processed to extract features of interest. In one embodiment, the extraction of features of interest may be accomplished by a machine leaning module (4) which may include an object of interest selection module components as detailed below.

Another aspect of the inventive methods and systems described herein may further include the step of generating a reference distribution by embedding the previously extracted features of interest from the reference sample, in this case a reference biological sample containing a microbe or cell population. In what follows, “embedding” refers to generic dimension reduction (also sometimes referred to as an “encoding”); the “embedding” can be accomplished via supervised techniques such as neural network embeddings calibrated by triplet-loss or unsupervised techniques like Principal Components Analysis (PCA) or extracted from the latent space representations of obtained by other unsupervised methods such as Variational Auto-Encoders (VAE) or Generative Adversarial Network (GAN); optionally with further dimension reduction via UMAP or t-SNE. As detailed below, this embedding process may convert the extracted features of interest to a lower dimensional feature set which can be used for classification or prediction. In another optional embodiment, one or more additional samples identified above may be utilized to generate additional reference distributions through the process of embedding the extracted features of interest from the images capture of the additional samples so as to again, convert the extracted features of interest to a lower dimensional feature set. In this preferred embodiment, the reference distributions of the reference's embedding, and optionally the additional embeddings of additional samples, may be defined by using a loss function to separate the embedded lower dimensional feature sets associated with each reference distribution. Further, the probability density of the individual extracted feature embeddings of the reference and optionally the additional samples may be estimated, and in a preferred embodiment, the probability density of one or more of the additional samples on the embedding space may be further estimated. In these embodiments, the low dimensional embeddings are obtained by altering the machine leaning module (4) output.

The high level of magnification offered by recently commercialized flow imaging microscopy instruments allows flow microscopes to record images of particles as small as 200 nm. The present inventors have discovered that this ability, when combined with ConvNets, can be used to image, detect and classify bacteria and other types of cells and particles, such as biomolecules. Thus, in one embodiment, the combination of HTI and ConvNets can be applied to detecting microbial infections of blood, urine or other biological samples (1). Current approaches for detecting blood infections rely predominantly on blood culture, a technique in which a blood sample is grown in media to promote microbial growth. If an organism grows in the media, the sample typically is tested using standard microbiological approaches to identify the type of microbe. This approach takes a significant amount of time in order to obtain a diagnosis; samples frequently require 24-48 hours for an organism to be culture to detectable levels and additional time to identify the pathogen. Additionally, this approach often requires large blood volumes (multiple mL) in order to reliably detect pathogens. These drawbacks are particularly significant for neonates who need rapid identification and treatment of any potential blood infections and from whom only <1 mL of blood may be drawn in order to diagnose an infection. HTI and ConvNets can be combined to detect microbial infections in approximately one hour of analysis with minimal blood volume from the patient.

In another embodiment, the invention includes novel systems, methods and compositions for early identification of microbial diseases, including, but not limited to, neonatal sepsis (and other septic conditions affecting all ages), urinary tract infections, septic arthritis, chorioamnionitis, microbial diseases of the brain and spinal cord, and microbial respiratory illness. The technique is also applicable to studying engineered cell lines. In the preferred embodiment shown in FIG. 1, the invention may include acoustic separation of microparticles, such as bacteria from a biological sample, application of high-resolution imaging, such as that provided by flow imaging microscopy to capture images of the separated biological sample, and the application of machine learning system to analyze the capture images.

Again, referring to FIG. 1, the invention includes a separation module (2) configured to separate the cells or other microparticles of a biological sample (1) by size. In one preferred embodiment, the invention includes a separation module (2) that enables the acoustic separation of cells and other microparticles in a biological sample (1) by size and/or compressibility. An exemplary acoustic separation module (2) may include, but not be limited to an AcouWash™ cell separation device manufactured by Acousort™. In this embodiment, the AcouWash™ or other acoustic separation module (2) may be used to process biological samples (1), such as blood, urine or sputum, generating a microbe rich sample, free of large host cells such as red and white blood cells that would otherwise clutter samples and make diagnostic techniques using imaging impossible.

The proposed strategy for detecting bloodstream infections utilizes flow imaging to image individual components, such as cells in a biological sample that have been process by a separation module (2) of the invention to separate cells or other microparticles of a biological sample (1) by size, preferably a blood or urine sample and apply machine learning systems as described herein to detect pathogenic cells within that sample (1). FIG. 1 generally illustrates an exemplary preferred embodiment using these two technologies to identify pathogenic cells in a biological sample (1) with roughly 1 hour of analysis time. In this embodiment, a biological sample, in the embodiment a blood sample, may be diluted with isotonic media and processed by a separation module (2) of the invention, and preferably an acoustic separation module and analyzed with a high-throughput image capture module (3), which may be preferably a HTI instrument capable of imaging particles smaller than 2 μm. Images potentially containing bacteria can then be isolated from the HTI data (1) by applying a combination of digital particle size filters and convolutional neural networks (ConvNets) to identify images of any remaining large blood cells (e.g., red and white blood cells) and smaller blood cells (e.g., platelets), respectively, and remove them from subsequent stages in the analysis. Once images potentially containing a microbe are isolated, the present inventors can use an additional ConvNet to predict an identity or species of the pathogen, as well as a characteristic of the microbe, such as its antibiotic-resistance. Finally, the present inventors may further use a final ConvNet trained via a fault detection, embodied in a fault detection module approach to estimate the confidence that the algorithm identified the correct pathogen in the previous step.

After the analysis is complete, this approach may return a diagnosis of sepsis or the presence of a bacterial infection as well as the type and characteristic of the bacteria responsible for the infection, such as anti-biotic resistance, Additionally, the approach yields images of any objects in the blood sample that were identified as potentially being pathogenic. These images give clinicians a method to check the raw data collected in the analysis before accepting the diagnosis and beginning treatment.

The primary benefits of this approach are its sensitivity to trace amounts of pathogenic cells even in small blood samples. Since HTI allows direct analysis of every cell in a blood sample, this approach can identify blood samples from a patient with a bloodstream infection or sepsis in cases where the sample only contains a few pathogenic cells. This sensitivity allows the inventive technology to accurately analyze even small blood samples such as those available from neonatal patients. Importantly, the sensitivity of this allows the elimination of the 24-48 culture step that is required with many other techniques for diagnosing bloodstream infections and instead look for pathogenic cells directly from the blood sample. While other techniques such as those based on flow cytometry or polymerase chain reactions (PCR) can also eliminate this culture step, many of these approaches rely on organism-specific labels or primers to achieve the sensitivity needed to detect pathogenic cells without relying on cell culture. The inventor's proposed approach does not require labeling to detect trace amounts of any pathogenic cells that may be in a given sample.

The sensitivity of the algorithm relaxes the amount of time and blood volume needed to perform the analysis. Each step of the proposed analysis can be performed quickly; sample preparation takes negligible time to perform, ConvNet analysis can be completed in a few seconds after the networks are trained, and HTI can be completed in one hour for a 50 μL blood sample. This novel approach can diagnose sepsis in approximately one hour—significantly faster than the 24-72 hours required for blood culture as well as the 4-8 hours required for many PCR-based approaches. Additionally, this approach does not require large blood samples from the patient to detect pathogenic species and is designed to give an accurate sepsis diagnosis even from a single drop of blood. The minimal volume and analysis time requirement make this approach ideal for diagnosing neonatal sepsis. Larger blood samples may also be analyzed using this approach, increasing the analysis time due to the extra volume but yielding more reliable detection of trace concentrations of the pathogen.

Again, referencing FIG. 1, the processed biological sample (1) may be further processed by a high-throughput image capture module (3). In a preferred embodiment, a high-throughput image capture module (3) may include a high-throughput imaging instrument that may allow for image capture of the individual cells or components of a processed biological sample (1). One non-limiting example of high-throughput imaging instrument may include a flow-imaging microscope.

As used herein, the terms “flow imaging microscopy” “high-throughput flow imaging,” “high-throughput flow imaging instrument,” “flow imaging instrument” “flow imaging,” “high-throughput microfluidic imaging device,” and “microfluidic imaging device,” are generally interchangeable interchangeably, refer to methods and instruments that allow the detection of objects in a high-throughput flow system. In certain embodiments, flow cytometric methods and instrumentation may fall under the broad category of HTI generally. As used specifically herein, the terms “high-throughput flow imaging microscopy instrument,” “HTI” or “flow imaging microscopy” or “high-throughput flow imaging instrument,” means any device, process or method that allows for the transport of particles, such as cells or bacteria, in a fluid, and in a preferred embodiment, transport of particles in a fluid through a microfluidic apparatus followed by an imaging step, and in a preferred embodiment microscopy imaging of said particles. Notably, a HTI device may include imaging that occurs dynamically as the fluid passes through the microfluidic device. Such dynamic flow imaging may include imaging devices that are known in the art, e.g., direct microscopy image capturing, classic and imaging flow cytometry, and exemplary devices such as a FlowCam Nano™ manufactured by Yokogawa Fluid Imaging Technologies, Inc., as well as devices and methods of Micro-Flow Imaging (MFI), which includes a particle analysis techniques using flow microscopy to quantify particles contained in a solution based on size. A HTI device may also include static imaging processes and devices, wherein a fluid, containing a quantity of particles, may be extracted and input into a medium where it is imaged, for example a fluid sample may be pipetted or otherwise input to a medium wherein the particles in the fluid are subsequently imaged by, for example, a HORIZON™ imaging system manufactured by Halo Labs, Burlingame, CA, or other similar device.

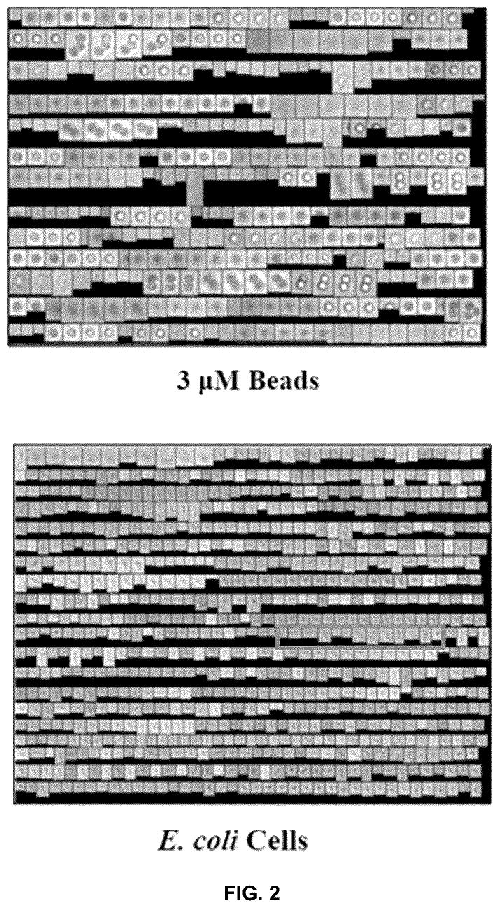

Referring to FIG. 1, the processed biological samples (1) may be imaged by high-throughput flow imaging instrument, and preferably a Flow Cam Nano™ instrument as described above, which is configured to capture high-resolution images of each cell from the processed biological sample (1) using oil immersion microscopy. As shown in FIG. 2, the image capture module (3) may be adapted to include an adapted high-throughput flow imaging instrument that takes multiple images of every cell from the processed biological sample (1), which can be process by the machine learning algorithm (4) as described below to obtain highly accurate results.

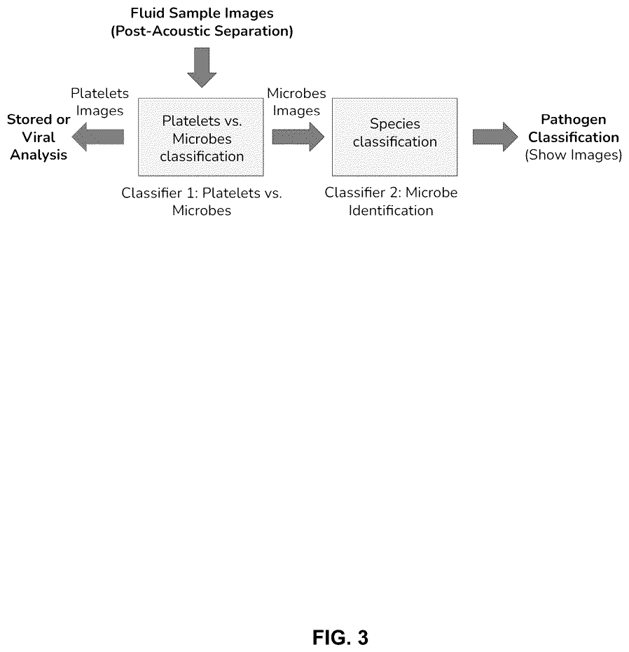

Referring again to FIG. 1, after obtaining a plurality of images from the image capture module (3), the images are process by a machine learning module (4) that is configured identify the presence of individual microparticles, such as cell and microbes, including the identification of microbial species as further described below. As outlined in FIG. 3, the machine learning module (4) may employ a multiple step classification process, and a sliding window sampler to make use of the image redundancy settings, resulting in highly accurate classification results. The machine learning module (4) may alternatively consist of an embedding obtained from a neural network trained in an unsupervised or supervised fashion.

As provided in the Examples below, the invention may include a variety of applications including, but not limited to: i) Diagnosis of Microbial Disease; ii) Cell Therapy Viability Analysis; iii) Determination of Pathogen Drug Resistance Characteristics; iv) Determination of Pathogen Degree of Toxicity; v) Real-time Monitoring of Treatment Success; vi) Real-time Monitoring of Developing Infections in Susceptible Patients; and vii) Determination of Infection Severity as measured by CFUs/mL.

In one embodiment, a plurality of images may be obtained to train and test machine learning module. In one embodiment, the machine learning module uses 50,000 training images, 20,000 test images, and 20,000 validation images, with a patience of 60, learning rate of 0.0001, and TensorFlow and Keras backends. Classification values for individual images may be used for a moving mean calculation, and confusion matrixes were generated using the resulting data.

As described above, the invention provides improved automated biological sample test systems for rapid analysis of target particles, such as biomolecules, such as cells and pathogens in biological samples processed through high-throughput cytometry or other similar separation or analysis methods. In preferred embodiments, these systems may rapidly and efficiently identify the presence of target particles, such as cells and biomolecules in a sample, and may further be used to analyze high volumes of biological samples without the need of human intervention.

The disclosed invention extends and modifies state-of-the-art technology in experimental high-throughput flow imaging microscopy, flow cytometry, machine learning, and computational statistics. The invention enables the ability to classify experimental images into pre-defined classes and/or label the observation as an a priori known or a priori unknown “fault” meaning that the observation is statistically unlikely to have come from a measured reference population of responses. As generally shown in FIG. 1, the invention may include a multi-component system to capture high-throughput flow imaging microscopy or separated biological samples and apply machine learning applications to such images and thereby achieve a classification of subject particles, cell, biomolecule or other target. Each of the modules in the diagram can be accomplished by a variety of methods and components.

In one preferred embodiment, the present inventors expand on the type of input and output of each module using terminology known by a person having ordinary or skill in the art. Notable, is that in the preferred embodiment demonstrated in FIG. 1, all of the parameters required to specify the function evaluations in the various modules may be assumed to have already been estimated using a large collection of labeled raw or processed image data (where “processed” implies that the modules upstream have produced the correct input) by minimizing a suitable “cost function”, where the cost function can aim at classification (e.g. a “cross entropy loss” function) as would be needed, for example, in pathogen analysis or the cost function can aim at developing a low dimensional representation through “image embeddings” for applications in fault detection (e.g. using a supervised triplet loss cost function or a least squares type reconstruction loss as used in unsupervised learning).

As shown in FIG. 1, a plurality of microscopy images may be generated by a high-throughput image capture module (3) that is tunable such that it is configured to capture multiple images of the same microparticle, such as a bacterium, such images being inputted into the inventive system for further analysis. In one preferred embodiment, a plurality of images may be captured of the individual components of a sample, such as a biological subjected to a HTI device. This high-throughput imaging may be further analyzed to detect, diagnose, and monitor harmful foreign infectious biomolecules, such as bacterium in mammals. In a preferred embodiment, microscopy images may be from a bright field or fluorescence microscope or other similar imaging device such as a HTI device. As will be discussed below, in preferred embodiments, a plurality of microscopy images may be used to generate training datasets. While the number of images required for such high-throughput training sets may depend on the application and feature of interest among other considerations, in one embodiment, such high-throughput training sets may range from at least 10³ to 10⁶ images, or more preferably 10⁴ to 10⁷ or more images.

In one preferred embodiment, a Machine learning module (4) may include a ConvNet feature extraction module may take a collection of raw or preprocessed (where the preprocessing step may cull images based on estimated size of objects in the image above or below a given size threshold that pass the physical pre-filtering step) images measured from a high-throughput microscopy device as input and extracts “features,” generally referred to as a “features of interest.” These features may typically be extracted via Convolutional Neural Networks (CNNs), but could be extracted by other feature extractors, such as Principal Component Analysis (PCA). The outputs of this module may be the resulting features and optionally the original image measurement for further processing downstream.

In another preferred embodiment, a Machine learning module (4) may include Fusion module that may be optionally used to leverage data and/or meta-information from other sources. The features from a ConvNet may be combined with other measurement or descriptive features through a variety of methods (e.g. a two input Artificial Neural Network, a Random Forest algorithm or Gradient Boosting algorithm for feature selection) producing a new set of feature of interest outputs or image embeddings; if there is no additional information to leverage or it is desired not to alter the features at this stage, this module can serve as an “identity” function producing output identical to all or a subset of the input to this module.

In another preferred embodiment, a Machine learning module (4) may include an object of interest selection module that may decide which measurements features and/or images may be further processed downstream and which will be ignored. For example, in a pathogen analysis embodiment, blood platelets from a processed biological sample (1) may be ignored in downstream analysis. In this embodiment silicone oil or air bubbles passing through a HTI instrument could also be ignored. This module can use another Artificial Neural Network (ANN) to produce a new set of features or embeddings (depending on the specific application) or can be a standard high-dimensional classifier acting on the input and serving as a “gate function.”

In alternative embodiments, this step can also be an “identity” function passing all or a subset of features through to the next step unaltered. The branch taken in the next step may be application dependent. One branch, which for example may be used in a pathogen identification embodiment, such that a machine learning module (4) may include one or more classification or classifier modules that assign a predefined label and probability of a class based on the passed in features/images using another classifier. The subsequent class and class probability output can either be the final output, or the features/raw input features can be embedded via another pretrained ANN and passed to the other branch, in this instance an optional fault detection module. The fault detection module,” as an optional part of the Machine learning module (4) may take low-dimensional embedding representations of the raw images and runs statistical hypothesis tests to check if it is statistically probable that the collection of embeddings has been drawn from a precomputed reference distribution of interest. This step may incorporate a precomputed empirically determined probability distribution (where the distribution function estimation can be parametric or nonparametric) of a suitable goodness-of-fit test statistic characterizing a large collection of labeled ground truth data. The aforementioned distribution may then be used to compute a p-value for each image in the “test dataset” enabling a user to detect if the test statistic generated by the collection of embeddings of the unlabeled data are statistically similar to the embeddings of the labeled reference distribution.

As further shown in FIG. 1, the output of the one or more classification modules of the Machine learning module (4) can be used to verify the diagnosis for the candidate predicted class label which may be useful in applications where a priori unanticipated contaminants of similar size to the objects of interest can be in the sample since the classification algorithm used in this stage is assumed to be trained on a fixed known list of candidate class labels.

Unless otherwise indicated, the method operations and device features disclosed herein involve techniques and apparatus used in microbiology, geometric optics, software design and programming, and statistics, which are within the skill of the art.

Unless defined otherwise herein, all technical and scientific terms used herein have the same meaning as commonly understood by one of ordinary skill in the art. Although any methods and materials similar or equivalent to those described herein find use in the practice or testing of the embodiments disclosed herein, some methods and materials are described in detail and represent preferred embodiments of the current inventive technology.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by an application, module, or both, which specifically includes cloud-based applications. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors or through a cloud-based application.

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the singular terms “a,” “an,” and “the” include the plural reference unless the context clearly indicates otherwise. The term “or” as used herein, refers to a non-exclusive or, unless otherwise indicated.

The terms defined immediately below are more fully described by reference to the specification as a whole. It is to be understood that this disclosure is not limited to the particular methodology, protocols, and reagents described, as these may vary, depending upon the context in which they are used by those of skill in the art.

The term “plurality” refers to more than one element. For example, the term is used herein in reference to more than one type of parasite or pathogen in a biological sample; more than one sample feature (e.g., a cell) in an image of a biological sample; more than one layer in a deep learning model; and the like.

The terms “threshold” herein refer to any number that is used as, e.g., a cutoff to classify a sample feature as particular type of parasite or pathogen, or a ratio of abnormal to normal cells (or a density of abnormal cells) to diagnose a condition related to abnormal cells, or the like. The threshold may be compared to a measured or calculated value to determine whether the source giving rise to such value suggests that it should be classified in a particular manner. Threshold values can be identified empirically or analytically. The choice of a threshold is dependent on the level of confidence that the user wishes to have to make the classification. Sometimes they are chosen for a particular purpose (e.g., to balance sensitivity and selectivity).

The term “biological sample,” or “sample” refers to a sample to be analyzed with the invention as generally described herein. In preferred embodiments, a “biological sample” or “sample” refers to a sample typically derived from a biological fluid, tissue, organ, etc., often taken from an organism suspected of having a condition, such as a disease or disorder, such as an infection. Such samples include, but are not limited to sputum/oral fluid, amniotic fluid, blood, a blood fraction, bone marrow, fine needle biopsy samples (e.g., surgical biopsy, fine needle biopsy, etc.), urine, semen, stool, vaginal fluid, peritoneal fluid, pleural fluid, tissue explant, organ culture, cell culture, and any other tissue or cell preparation, or fraction or derivative thereof or isolated therefrom. In addition, as generally used herein a “biological sample” or “sample” may include any sample that may be subject to a high-throughput process, such as high throughput flow imaging microscopy.

A “reference sample” as used herein is a sample that may be used to train a computer learning systems, such as by generating a training dataset. A “test sample” as used herein is a sample that may be used to generate a test dataset, for example of one or more features of interest, which may be qualitatively and/or quantitatively compared to a training dataset as generally described herein.

A biological sample may be taken from a multicellular organism or it may be of one or more single cellular organisms. In some cases, the biological sample is taken from a multicellular organism, such as a mammal, and includes both cells comprising the genome of the organism and cells from another organism such as a parasite or pathogen. The sample may be used directly as obtained from the biological source or following a pretreatment to modify the character of the sample. For example, such pretreatment may include preparing plasma from blood, diluting viscous fluids, culturing cells or tissue, and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. Such “treated” or “processed” samples are still considered to be biological samples with respect to the methods described herein. In one preferred embodiment, a “processed biological sample,” or “processed sample” refers to a biological sample that has been processed by a separation module (2) of the invention, and preferably an acoustic separation device of the invention, or otherwise undergone a separation step to separate differently sized objects in the sample.

Biological samples can be obtained from any subject or biological source. Although the sample is often taken from a human subject (e.g., a patient), samples can be taken from any organism, including, but not limited to mammals (e.g., dogs, cats, horses, goats, sheep, cattle, pigs, etc.), non-mammal higher organisms (e.g., fish, reptiles, amphibians), vertebrates and invertebrates, and may also be or include any single-celled organism such as a eukaryotic organism (including plants and algae) or a prokaryotic organism, archaeon, microorganisms (e.g. bacteria, archaea, fungi, protists, viruses), and aquatic plankton.

In various embodiments described herein, a biological sample is taken from an individual or “host.” Such samples may include any of the cells of the host (i.e., cells having the genome of the individual) or host tissue along with, in some cases, any non-host cells, non-host multicellular organisms, etc. described below. In various embodiments, the biological sample is provided in a format that facilitates imaging and automated image analysis. As an example, the biological sample may be stained before image analysis.

As used herein, a host is an organism providing the biological sample. Examples include higher animals including mammals, including humans, reptiles, amphibians, and other sources of biological samples as presented above.

As used herein, a “feature,” “feature of interest” or “sample feature” is a feature of a sample that represents a quantifiable and/or observable feature of an object or particle passing through a high-throughput system, and preferably a feature of a prokaryotic organism in a biological sample. In certain embodiments, a “feature of interest” may potentially correlate to a clinically relevant condition. In certain embodiments, a feature of interest is a feature that appears in an image of a sample, such as a biological sample, and may be recognized, segmented, and/or classified by a machine learning module (3). A feature of interest presented above can be used as a separate classification for the machine learning systems described herein. Such systems can classify any of these alone or in combination with other examples.

As used herein, a machine learning system or model is a trained computational model that takes a feature of interest, such as cellular artifacts extracted from an image and classifies them as, for example, particular cell types, parasites, or bacteria. Cellular artifacts that cannot be classified by the machine learning model are deemed peripheral or unidentifiable objects. Examples of machine learning models include neural networks, including recurrent neural networks and convolutional neural networks; random forests models, including random forests; restricted Boltzmann machines; recurrent tensor networks; and gradient boosted trees. The term “classifier” (or classification model) is sometimes used to describe all forms of classification model including deep learning models (e.g., neural networks having many layers) as well as random forests models.

As used herein, a machine learning system may include a deep learning model that may include a function approximation method aiming to develop custom dictionaries configured to achieve a given task, be it classification or dimension reduction. It may be implemented in various forms such as by a neural network (e.g., a convolutional neural network), etc. In general, though not necessarily, it includes multiple layers. Each such layer includes multiple processing nodes and the layers process in sequence, with nodes of layers closer to the model input layer processing before nodes of layers closer to the model output. In various embodiments, one-layer feeds to the next, etc. The output layer may include nodes that represent various classifications. In some embodiments, a deep learning model is a model that takes data with very little preprocessing, although it may be segmented data such as cellular artifact, or other features of interest may be extracted from an image, and outputs a classification of the cellular artifact.

In various embodiments, a deep learning model may have significant depth and can classify a large or heterogeneous array of features of interest, such as particles in a liquid suspension, or cellular artifacts, such as pathogens or gene expression. In some contexts, the term “deep” means that model has a plurality of layers of processing nodes that receive values from preceding layers (or as direct inputs) and that output values to succeeding layers (or the final output). Interior nodes are often “hidden” in the sense that their input and output values are not visible outside the model. In various embodiments, the operation of the hidden nodes may not be monitored or recorded during operation. The nodes and connections of a deep learning model can be trained, for example with a “reference” or “additional sample,” and retrained without redesigning their number, arrangement, interface with image inputs, etc. and yet classify a large heterogeneous range of features of interest, such as cells, microorganisms, cells expressing one or more genes, or microorganisms that may have a phenotypic or genotypic traits, such as antibiotic resistance.

In various aspects, provided herein are systems and methods for identifying and optionally characterizing a feature of interest, by analyzing the feature of interest from a test sample and thereby generating a test dataset and comparing it to a training dataset generated from a reference sample, and optionally one or more additional samples. A feature of interest in this embodiment may include a feature of the cell, such as cell morphology among others.

For example, in one specific embodiment, provided herein are systems and methods for identifying and optionally characterizing a cell of interest as a target cell by analyzing a signature of the cell of interest, quantified by a “feature of interest” extracted from the image via a ConvNet, in a test sample and comparing it to a signature of the target cell from a reference sample. A signature of a cell, or “feature of interest” may also include a physical feature of the cell, such as cell morphology, as well as the presence, absence, or relative amount of gene expression within and/or associated with the cell, a phenotypic or genotypic traits, such as antibiotic resistance in a microorganism.

A “feature of interest” of a cell of interest may be useful for diagnosing or otherwise characterizing a disease or a condition in a patient from which the potential target cell was isolated. As used herein, an “isolated cell” refers to a cell separated from other material in a biological sample using any separation method, and preferably a separation module (2) of the invention. An isolated cell may be present in an enriched fraction from the biological sample, and thus its use is not meant to be limited to a purified cell. In some embodiments, the morphology of an isolated cell is analyzed. For target cells indicative of infection, analysis of a cell signature is useful for a number of methods including diagnosing infection, determining the extent of infection, determining a type of infection, and monitoring progression of infection within a host or within a given treatment of the infection. Some of these methods may involve monitoring a change in the signature of the target cell, which includes an increase and/or decrease, and/or any change in morphology.

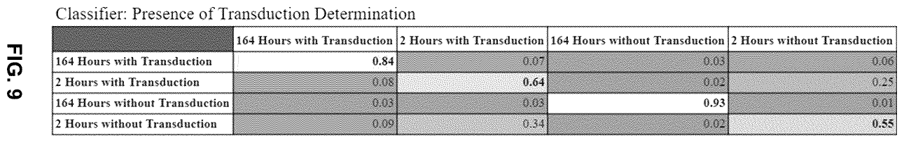

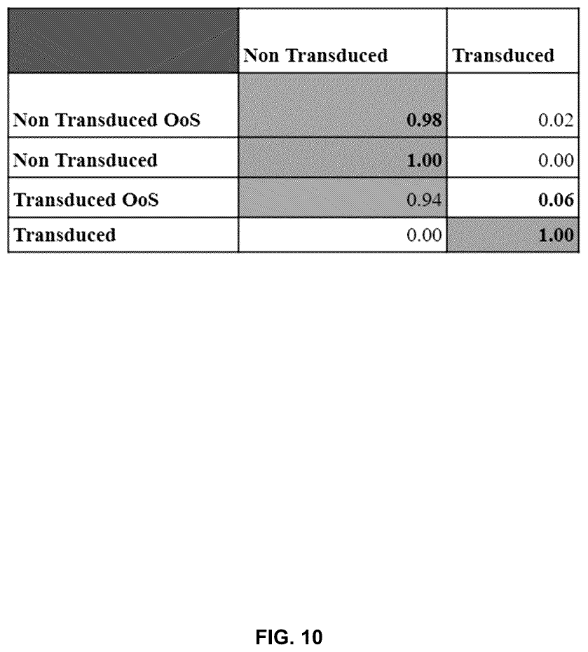

In some embodiments, a “feature of interest” of a cell of interest is analyzed in a fraction of a biological sample of a subject, wherein the biological sample has been processed to enrich for a target cell. In some cases, the enriched fraction lacks the target cell and the absence of a signature of a target cell in the enriched fraction indicates this absence. Target cells include, for example transduced T-cells used to form CAR-T cells and non-transduced T-cells as demonstrated in FIG. 9 or FIG. 10.

In some embodiments, a “Population Distribution” refers to an aggregate collection of features of interest associated with a reference or other sample as generally described herein. The “Population Distribution” corresponds to the unknowable cumulative distribution function characterizing a population. This quantity is estimated via the probability density function in some embodiments.

As used herein, “Target Cell Populations” refers to the identified target cells in aggregate form. These populations can be thought of as point clouds that display characteristic shapes and have aggregate locations in a multidimensional space. In the multidimensional space, an axis is defined by a flow measurement channel, which is a source of signal measurements in flow cytometry. Signals measured, for example, in flow cytometry may include, but are not limited to, optical signals and measurements. Exemplary channels of optical signals include, but are not limited to, one or more of forward scatter channels, side scatter channels, and laser fluorescence channels.

All flow cytometry instrument channels or a subset of the channels described herein may be used for the axes in the multidimensional space. A population of cells may be considered to have changed in the multidimensional channel space when the channel values of its individual cell members change and in particular when a large number of the cells in the population have changed channel values. For example, the point cloud representing a population of cells can be seen to vary in location on a 2-dimensional (2D) dot plot or intensity plot when samples are taken from the same individual at different times. Similarly, the point cloud representing a population of cells can shift, translate, rotate, or otherwise change shape in multidimensional space. Whereas conventional gating provides total cell count within a gate region, the location and other spatial parameters of certain cell population point clouds in multidimensional space, in addition to providing total cell count, provide additional information which can also be used distinguish between normal subjects (e.g., subjects without an infection) and infected patients (e.g., subjects with a parasite or pathogen infection).

Provided herein are systems and methods for identifying and optionally characterizing a cell, cells of interest as a target cell by analyzing a signature of the cell of interest. In some instances, a cell of interest is a parasitic or pathogenic cell. Flow cytometry may be used to measure a signature of a cell such as the presence, absence, or relative amount of the cell, or through differentiating physical or functional characteristics of the target cells of interest. Cells of interest identified using the systems and methods as described herein include cell types implicated in a disease, disorder, or a non-disease state. Exemplary types of cells include, but are not limited to, parasitic or pathogenic cells, infecting cells, such as bacteria, viruses, fungi, helminths, and protozoans. Cells of interest in some cases are identified by at least one of alterations in cell morphology, cell volume, cell size and shape, as well as other phenotypic or genotypic traits, such as antibiotic resistance.

In some instances, cells are acquired from a subject by a blood draw, a marrow draw, or a tissue extraction. Often, cells are acquired from peripheral blood of a subject. Sometimes, a blood sample is centrifuged using a density centrifugation to obtain mononuclear cells, erythrocytes, and granulocytes. In some instances, the peripheral blood sample is treated with an anticoagulant. In some cases, the peripheral blood sample is collected in, or transferred into, an anticoagulant-containing container. Non-limiting examples of anticoagulants include heparin, sodium heparin, potassium oxalate, EDTA, and sodium citrate. Sometimes a peripheral blood sample is treated with a red blood cell lysis agent.

Alternately or in combination, cells are acquired by a variety of other techniques and include sources such as bone marrow, ascites, washes, and the like. In some cases, tissue is taken from a subject using a surgical procedure. Tissue may be fixed or unfixed, fresh or frozen, whole or disaggregated. For example, disaggregation of tissue occurs either mechanically or enzymatically. In some instances, cells are cultured. The cultured cells may be developed cell lines or patient-derived cell lines. Procedures for cell culture are commonly known in the art.

Systems and methods as described herein can involve analysis of one or more test samples from a subject compared against one or more reference samples/datasets. A sample may be any suitable type that allows for the analysis of different discrete populations of cells. A sample may be any suitable type that allows for analysis of a single cell population. Samples may be obtained once or multiple times from a subject. Multiple samples may be obtained from different locations in the individual (e.g., blood samples, bone marrow samples, and/or tissue samples), at different times from the individual (e.g., a series of samples taken to diagnose a disease or to monitor for return of a pathological condition), or any combination thereof. These and other possible sampling combinations based on sample type, location, and time of sampling allow for the detection of the presence of cells before and/or after infection and monitoring for disease.

When samples are obtained as a series, e.g., a series of blood samples obtained after treatment, the samples may be obtained at fixed intervals, at intervals determined by status of a most recent sample or samples, by other characteristics of the individual, or some combination thereof. For example, samples may be obtained at intervals of approximately 1, 2, 3, or 4 days, at intervals of approximately 1, 2, 3, 4, 5, 6, 7, 8, 9, 10, or 11 hours, at intervals of approximately 1, 2, 3, 4, 5, or more than 5 months, or some combination thereof.

To prepare cells for analysis using the methods and systems described herein, cells can be prepared in a single-cell suspension. For adherent cells, both mechanical or enzymatic digestion and an appropriate buffer can be used to remove cells from a surface to which they are adhered. Cells and buffer can then be pooled into a sample collection tube. For cells grown in suspension, cells and medium can be pooled into a sample collection tube. Adherent and suspension cells can be washed by centrifugation in a suitable buffer. The cell pellet can be re-suspended in an appropriate volume of suitable buffer and passed through a cell strainer to ensure a suspension of single cells in suitable buffer. The sample can then be vortexed prior to performing a method using the flow cytometry system on the prepared sample.

Once cell samples have been collected, they may be processed and stored for later usage, processed and used immediately, or simply used immediately. In some cases, processing includes various methods of treatment, isolation, purification, filtration, or concentration. In some instances, fresh or cryopreserved samples of blood, bone marrow, peripheral blood, tissue, or cell cultures can be used for flow cytometry. When samples are stored for later usage, they may be stabilized by collecting the sample in a cell preparation tube and centrifuging the tube after collection.

When samples are obtained as a series, e.g., a series of blood samples obtained after treatment, the samples may be obtained at fixed intervals, at intervals determined by status of a most recent sample or samples, by other characteristics of the individual, or some combination thereof. For example, samples may be obtained at intervals of approximately 1, 2, 3, or 4 days, at intervals of approximately 1, 2, 3, 4, 5, 6, 7, 8, 9, 10, or 11 hours, at intervals of approximately 1, 2, 3, 4, 5, or more than 5 months, or some combination thereof.

To prepare cells for analysis using the methods and systems described herein, cells can be prepared in a single-cell suspension. For adherent cells, both mechanical or enzymatic digestion and an appropriate buffer can be used to remove cells from a surface to which they are adhered. Cells and buffer can then be pooled into a sample collection tube. For cells grown in suspension, cells and medium can be pooled into a sample collection tube. Adherent and suspension cells can be washed by centrifugation in a suitable buffer. The cell pellet can be re-suspended in an appropriate volume of suitable buffer and passed through a cell strainer to ensure a suspension of single cells in suitable buffer. The sample can then be vortexed prior to performing a method using the flow cytometry system on the prepared sample.

Once cell samples have been collected, they may be processed and stored for later usage, processed and used immediately, or simply used immediately. In some cases, processing includes various methods of treatment, isolation, purification, filtration, or concentration. In some instances, fresh or cryopreserved samples of blood, bone marrow, peripheral blood, tissue, or cell cultures are used for flow cytometry. When samples are stored for later usage, they may be stabilized by collecting the sample in a cell preparation tube and centrifuging the tube after collection.

A feature of interest can be detected by any one or more of various methods generally referred to a high-throughput imaging (HTI) system. The term HTI, as used generally herein refers to methods and instruments that allow the detection of objects in a high-throughput microparticle imaging system. In certain embodiments, flow cytometric methods and instrumentation may fall under the broad category of HTI generally.

HTI is capable of characterizing complex images of single subvisible particles. In HTI embodiments, a small liquid sample is pumped through a microfluidic flow-cell, and a digital microscope is used to record upwards of 10\hat{ }6 images of individual particles, such as microbes in a single experiment. A rich amount of information is encoded in this image data. HTI analysis methods to date have depended on a small number of “morphological features” (such as aspect ratio, compactness, intensity, etc.) in order to characterize the single particle images, but this short list of features (often containing highly correlated quantities) neglects a great deal of information contained in the full (RGB or grayscale) HTI images. Deep convolutional neural networks (CNNs or “ConvNets”) along with supervised or semi-supervised learning, as described herein may harness the large amount of complex digital information encoded in images and automatically extract the relevant features of interest for a given classification or fault detection task without requiring the selection, labeling, or specification of “morphological features”. In a preferred embodiment utilizing HTI, brightfield, or other microscopy images are captured in successive frames as a continuous sample stream passes through a flow cell centered in the field-of-view of a custom magnification system having a well-characterized and extended depth-of-field. HTI allows not only enumerating the subvisible particles present in the sample, but also visual examination of the images of all captured particles. A standard bench-top Micro-Flow Imaging (MFI) configuration uses a simple fluidics system, where sample fluid is drawn either directly from a pipette tip or larger container through the flow cell using a peristaltic pump. The combination of system magnification and flow-cell depth determines the accuracy of concentration measurement. Concentration and parameter measurements are absolute but may be re-verified using particle standards. Typical sample volumes range from <0.25 to tens of milliliters. Frame images displayed during operation provide immediate visual feedback on the nature of the particle population in the sample. The digital images of the particles or cells present in the sample may be analyzed using image morphology analysis software that allows quantification in size and count. This system software can extract particle images using a sensitive threshold to identify pixel groups which define each particle. Successive frames, each containing many particle images, are analyzed in real time. Maximum instrument sensitivity for detecting near-transparent particles is achieved by automatically optimizing threshold values, using low-noise electronics, implementing noise reduction algorithms, and compensating for all possible non-uniformities in spatial and pulse-to-pulse illumination. Ten-bit grayscale resolution may be used to improve threshold accuracy. Images may be analyzed to compile a database containing count, size, concentration, as well as a range of shape and image contrast parameters. This database may be interrogated by the computer's application software to produce parameter distributions using histograms and scatter plots. The software supports image filtering by calculating a trial filter based on user selected representative particles and then interacting with the user to optimize this filter to extract similar particles from the total population. This feature allows particle sub-populations to be isolated and independently analyzed. Particle images are available for verification, further investigation, and analysis. Once a successful assay has been developed and validated, the resulting protocol, including run parameters, software filters, and report formats, can be saved for future use.

Direct imaging particle measurement technologies such as HTI have a number of advantages over indirect obscuration or scattering-based measurements. For example, they do not rely on a correlation between particle size and the magnitude of a scattered or obscured optical signal as calibrated using polystyrene reference beads. Provided the contrast in the particle image is sufficient for the pixels to be resolved by the system threshold, the particle will be detected and measured. No calibration by the user is required. The particle images captured by the system also provide qualitative and quantitative information about the target particle population. Qualification studies based on National Institute of Standards and Technology-traceable polystyrene beads have shown that the technology can meet high standards for sizing, concentration accuracy, and repeatability.

Non-limiting examples of commercially available HTI instruments suitable for use in the systems and methods of this disclosure include Sysmex Flow Particle Image Analyzer (FPIA) 3000 and the Morphologically-Directed Raman Spectroscopy (MDRS), Raman signaling being one preferred modality of an image singal among others, by Malvern Instruments (Worcestershire, UK), various Occhio Flowcell systems by Occhio (Angleur, Belgium), the MicroFlow Particle Sizing System by JM Canty (Buffalo, NY, USA), several MFI systems by ProteinSimple (Santa Clara, CA, USA), various Flow Cytometer and Microscopes, e.g. FlowCAM™, systems by Fluid Imaging (Yarmouth, ME, USA), and Backgrounded Membrane Imaging systems, e.g. HORIZON™ and Aura™, by Halo Labs (Burlingame, CA, USA).

In certain embodiments, machine learning systems may include artificial neural networks (ANNs) which are a type of computational system that can learn the relationships between an input data set and a target data set. ANN name originates from a desire to develop a simplified mathematical representation of a portion of the human neural system, intended to capture its “learning” and “generalization” abilities. ANNs are a major foundation in the field of artificial intelligence. ANNs are widely applied in research because they can model highly non-linear systems in which the relationship among the variables is unknown or very complex. ANNs are typically trained on empirically observed data sets. The data set may conventionally be divided into a training set, a test set, and a validation set.

In supervised learning applications, the labeled data is used to form an objective function (e.g., cross-entropy loss, “triplet” loss, “Siamese” loss, or custom loss functions encoding physical information). The network parameters are updated to optimize the specified loss function. In particular, a type of neural network called a feed-forward back-propagation classifier can be trained on an input data set to generate feature representations minimizing the cost function over the training samples. Variants of stochastic gradient descent are often used to search parameter space in combination with the back-propagation algorithm to minimize the cost function specified over the training data inputs. After a large number of training iterations, the ANN parameter updates may be stopped; the stopping criteria typically leverages evaluations of the network on the validation data set (the other stopping criteria can be applied).

The goal of training a neural network is typically to have the ANN make an accurate prediction of a new sample, for example, a sample not used during training or validation. Accuracy of the prediction is often measured against the objective function, for example, classification accuracy may be enabled by providing the truth label for the new sample. However, in one embodiment of the present inventor's method, is the use of neural networks for embedding/dimension reduction, namely takes a set large number of pixels in a source HTI image, and summarize the information content with low (2-256) dimensional feature output embedding values from the ANN; the feature embedding can be reduced to 2-6 via post-processing techniques like t-SNE or UMAP; the statistical distribution of the 2-6 dimensional embedding point cloud is determined by nonparametric methods, and the proximity of a new set of sample “test points” is statistically tested via suitable and appropriate hypothesis tests, for example Kolmogorov-Smirnov tests, Hong and Li's Rosenblatt transform based test or Copula transform based goodness-of-fit approaches.

ANNs have been applied to a number of problems in medicine, including image analysis, biochemical analysis, drug design, and diagnostics. ANNs have recently begun to be utilized for medical diagnostic problems. ANNs have the ability to identify relationships between patient data and disease and generate a diagnosis based exclusively on objective data input to the ANN. The input data will typically consist of symptoms, biochemical analysis, and other features such as age, sex, medical history, etc. The output will consist of the diagnosis.

Disclosed herein is a novel method that presents the unprocessed HTI image data to a machine learning system, such as an ANN for analysis that provides diagnostic, prognostic, and fault detection.

Many types of machine learning models may be employed in embodiments of inventive technology. In general, such models take as inputs one or more features of interest, such as cellular artifacts extracted from an image of a sample pass through a high-throughput system, and, with little or no additional preprocessing, they classify individual feature of interest as particular cell types, parasites, pathogens, health conditions, etc. without further intervention. In alternative embodiments, such models take as inputs one or more features of interest, such as bacterial size, or morphology or an antibiotic resistance trait, and, with little or no additional preprocessing, they classify individual artifacts as particular biomolecule type or characteristics, such as protein aggregation. Typically, the inputs need not be categorized according to, for example their morphological or other features for the machine learning model to classify them.

Two primary embodiments of machine learning modules (4) of the invention may include “deep” convolutional neural network (ConvNet) models and a randomized Principal Component Analysis (PCA) random forests model. However, other forms machine learning model may be employed in the context of this disclosure. A random forests model is relatively easy to generate from a training dataset and may employ relatively fewer training set members. A convolutional neural network may be more time-consuming and computationally expensive to generate from a training set, but it tends to be better at accurately classifying features of interest, such as cellular artifacts or protein aggregates.

Typically, whenever a parameter of the processing system is changed, the deep learning model is retrained. Examples of changed parameters include sample (e.g., blood) acquisition and processing, HTI instrumentation, image acquisition components, etc. Due to the machine learning based nature of the classification techniques, it is possible to upload training samples, also referred generally to as reference samples of, for example, dozens of other parasite, or pathogen HTI images, and immediately have the model ready to identify new cell types and/or conditions.

Certain aspects of the inventive technology provide a system and method for identifying a sample feature of interest in a sample, such as a biological sample of a host organism. In some embodiments, the sample feature of interest is associated with a disease. The system includes a HTI instrument to capture digital images of the biological sample and one or more processors communicatively connected to an image capturing device, such as a camera-which may be part of a HTI instrument in some embodiments. In some embodiment, the one or more processors of the system are configured to perform a method for identifying a sample feature of interest. In some embodiments, the one or more processors of the system are configured to receive the one or more images of the biological sample captured by the HTI instrument. The one or more processors are optionally configured to segment the one or more images of the biological sample to obtain a plurality of images of the individual components of the sample passing through, in this embodiment a high-throughput HTI instrument.

In some embodiments, a segmentation operation may be applied which may include converting the one or more images of the biological sample from color images to grayscale images. Various methods may be used to convert the one on one or more images from color images to grayscale images. In some embodiments, the grayscale images are further converted to binary images using an Otsu thresholding method.

In some embodiments, the binary images may be transformed using a using a Euclidean distance transformation method as further described elsewhere herein. In some embodiments, the segmentation further involves identifying local minima of pixel values obtained from the Euclidean distance transformation. The local minima of pixel values indicate central locations of potential cellular artifacts. In some embodiments, the segmentation operation also involves applying a Sobel filter to the one or more images of the biological sample. In some embodiments, the gray scale images are used. Data obtained through the Sobel filter accentuate edges of potential cellular artifacts.

In some embodiments, segmentation further involves splicing the one or more images of the biological sample using the local maxima and data obtained from applying the Sobel filter, thereby obtaining a plurality of images of the cellular artifacts. In some applications, each spliced image includes a cellular artifact. In some embodiments, the splicing operation is performed on color images of the biological sample, thereby obtaining a plurality of images of the cellular artifacts in color. In other embodiments, gray scale images are spliced and used for further classification analysis.

In some embodiments, each of the plurality of images of the cellular artifacts is provided to a machine-learning classification system to classify a feature of interest. In some embodiments, the machine-learning system includes a neural network model. In some embodiments, the neural network model includes a convolutional neural network model. In some embodiments, the machine-learning classification model includes a principal component analysis and a Random Forests classifier.

In some embodiments where the machine-learning system includes principal component analysis and a random forests classifier, each of the plurality of images of the feature of interest, such as a cellular artifact, is standardized and converted into, e.g., a 50×50 matrix, each cell of the matrix being based on a plurality of image pixels corresponding to the cell. This conversion helps to reduce the total amount of data to be analyzed. Different matrix sizes can be used depending on the desired computational speed and accuracy.

The system may include two or more modules in addition to a segmentation module. For example, images of individual features of interest may be provided by the segmentation module to two or more machine learning modules, each having its own classification characteristics. In certain embodiments, machine learning modules are arranged serially or pipelined. In such embodiments, a first machine learning module receives individual features of interest and classifies them coarsely. A second machine learning module receives some or all of the coarsely classified features of interest and classifies them more finely.

As mentioned, the reduced data of the plurality of images of the cellular artifacts may undergo dimensional reduction using, e.g., PCA. In some embodiments, the principal component analysis includes randomized principal component analysis. In some embodiments, about twenty principle components are obtained. In some embodiments, about ten principal components are obtained from the PCA. In some embodiments, the obtained principal components are provided to a random forests classifier to classify the cellular artifacts.

In certain embodiments, a system having a neural network, e.g., a convolutional neural network, takes as input the pixel data of cellular artifacts extracted through segmentation. The pixels making up the cellular artifact are divided into slices of predetermined sizes, with each slice being fed to a different node at an input layer of the neural network. The input nodes operate on their respective slices of pixels and feed the resulting computed outputs to nodes on a next layer of the neural network, which layer is deemed a hidden layer of the neural network. Values calculated at the nodes of this second layer of the network are then fed forward to a third layer of the neural network where the nodes of the third layer act on the inputs they receive from the second layer and generate new values which are fed to a fourth layer. The process continues layer-by-layer until values reach an output layer containing nodes representing the separate classifications for the input cellular artifact pixels. After execution of the classification, each of the output nodes may be probed to determine whether the output is true or false. A single true value classifies the input cellular artifact.

Typically, the various layers of a convolutional neural network correspond to different levels of abstraction associated with the classification process. For example, some inner layers may correspond to classification based on a coarse outer shape of a feature of interest, such as a cellular artifact, for example circular, non-circular ellipsoidal, sharp angled, etc., while other inner layers may correspond to a different aspect or separate feature of interest, such as the texture of the interior of the cellular artifact, a smoothness of the perimeter of the cellular artifact, etc. In general, a plurality of rules governing which layers conduct which particular aspects of the classification process may be implemented. The training of the neural network may simply define nodes and connections between nodes such that the model more accurately classifies a feature of interest like cellular artifacts from an image of a biological sample.

Deep convolutional neural networks may include multiple feed forward layers. As known to those of skill in the art, these layers aim to extract relevant features from an input image; the features extracted depend on the objective function used for training. The convolutional layer's parameters include a set of learnable filters (or kernels), which have a small receptive field, but are applied to the entire input image region in the convolution step. In certain embodiments, during the forward pass, each filter is convolved across the width and height of the input image, computing a type of dot product between the entries of the filter and the input and producing an activation map associated with that filter. As a result, the network learns filters that activate when they encounter some specific type of feature at some spatial position in the input. The resulting activation maps are processed in both standard feed forward fashion and using “skip connections” in conjunction with feed forward output.

Convolutional networks may include local or global pooling layers, which reduce the dimensionality of the activation maps. They also include various combinations of convolutional, fully connected layers, skip connections, and customized layers, for example squeeze excite, residual blocks, or spatial transformer subnetworks. The neural network may include various combinations of feed forward stacked layers in order to generate feature representations of the input image data. The specific nature of the estimated features (obtained via supervised or unsupervised training) depends on the objective function, the input data, and the neural network architecture selected.

In certain embodiments, the deep learning image classification model may employ TensorFlow. Routines available from Google of Mountain View, Calif. or may employ PyTorch routines available from Facebook of Menlo Park, Calif. Some embodiments may employ VGG style network architectures, Google's simplified Inception net architecture, or Residual Networks, or multiscale Dilated Residual Networks (DRN). Modules like the Squeeze Excite or Spatial Transformer subnetworks may be inserted in the aforementioned networks using standard loss or custom loss functions.

The embodiments disclosed herein may be implemented as a system for topographical computer vision through automatic imaging, analysis and classification of physical samples using machine learning techniques and/or stage-based scanning. Any of the computing systems described herein, whether controlled by end users at the site of the sample or by a remote entity controlling a machine learning model, can be implemented as software components executing on one or more general purpose processors or specially designed processors such as programmable logic devices (e.g., Field Programmable Gate Arrays (FPGAs)) and/or Application Specific Integrated Circuits (ASICs) designed to perform certain functions or a combination thereof. In some embodiments, code executed during operation of image acquisition systems and/or machine learning models (computational elements) can be embodied by a form of software elements which can be stored in a nonvolatile storage medium (such as optical disk, flash storage device, mobile hard disk, cloud-based systems etc.), including a number of instructions for making a computer device (such as personal computers, servers, network equipment, etc.). Image acquisition algorithms, machine learning models and/or other computational structures described herein may be implemented on a single device or distributed across multiple devices. The functions of the computational elements may be merged into one another or further split into multiple sub-modules.

The hardware device can be any kind of device that can be programmed including, for example, any kind of computer including smart mobile devices (watches, phones, tablets, and the like), personal computers, powerful servers or supercomputers, or the like. The device includes one or more processors such as an ASIC or any combination processors, for example, one general purpose processor and two FPGAs. The device may be implemented as a combination of hardware and software, such as an ASIC and an FPGA, or at least one microprocessor and at least one memory with software modules located therein. In various embodiments, the system includes at least one hardware component and/or at least one software component. The embodiments described herein could be implemented in pure hardware or partly in hardware and partly in software. In some cases, the disclosed embodiments may be implemented on different hardware devices, for example using a plurality of CPUs equipped with GPUs capable of accelerating scientific computation.

Each computational element may be implemented as an organized collection of computer data and instructions. In certain embodiments, an image acquisition algorithm and a machine learning model can each be viewed as a form of application software that interfaces with a user and with system software. System software typically interfaces with computer hardware, typically implemented as one or more processors (e.g., CPUs or ASICs as mentioned) and associated memory. In certain embodiments, the system software includes operating system software and/or firmware, as well as any middleware and drivers installed in the system. The system software provides basic non-task-specific functions of the computer. In contrast, the modules and other application software are used to accomplish specific tasks. Each native instruction for a module is stored in a memory device and is represented by a numeric value.

At one level a computational element is implemented as a set of commands prepared by the programmer/developer. However, the module software that can be executed by the computer hardware is executable code committed to memory using “machine codes” selected from the specific machine language instruction set, or “native instructions,” designed into the hardware processor. The machine language instruction set, or native instruction set, is known to, and essentially built into, the hardware processor(s). This is the “language” by which the system and application software communicates with the hardware processors. Each native instruction is a discrete code that is recognized by the processing architecture and that can specify particular registers for arithmetic, addressing, or control functions; particular memory locations or offsets; and particular addressing modes used to interpret operands. More complex operations are built up by combining these simple native instructions, which are executed sequentially, or as otherwise directed by control flow instructions.

The inter-relationship between the executable software instructions and the hardware processor may be structural. In other words, the instructions per se may include a series of symbols or numeric values. They do not intrinsically convey any information. It is the processor, which by design was preconfigured to interpret the symbols/numeric values, which imparts meaning to the instructions.

In certain embodiments, the modules or systems generally used herein may be configured to execute on a single machine at a single location, on multiple machines at a single location, or on multiple machines at multiple locations. When multiple machines are employed, the individual machines may be tailored for their particular tasks. For example, operations requiring large blocks of code and/or significant processing capacity may be implemented on large and/or stationary machines not suitable for mobile or field operations. Such operations may be implemented on hardware remote from the site where the sample is processed, for example on a server or server farm connected by a network to a field device that captures the sample image, or through a cloud-based network. Less computationally intensive operations may be implemented on a portable or mobile device used in the field for image capture.

Various divisions of labor are possible: for example, a mobile device used in the field may contain processing logic to coarsely discriminate between leukocytes, erythrocytes, and pathogens, and optionally to provide counts for each of these. In some cases, the processing logic includes image capture logic, segmentation logic, and course classification logic, with the latter optionally implemented as a random forest model. These logic components may be implemented as relatively small blocks of code that do not require significant computational resources.

Logic that executes remotely (e.g., on a remote server or even supercomputer) discriminates between different types of leukocytes. As an example, such logic can classify eosinophils, monocytes, lymphocytes, basophils, and neutrophils. Such logic may be implemented as a deep learning convolutional neural network and require relatively large blocks of code and significant processing power. With the leukocytes or parasites or pathogens correctly identified, the system may additionally execute differential models for diagnosing conditions based on differential amounts of various combinations of the five leukocyte types.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1. Acoustic Separation of Biological Sample Components

In one embodiment, the initial step of the process includes the acoustic separation of the biological samples using the separation module, which in this embodiment includes the Acouwash™ Instrument. During this separation the small cells and particles contained in the sample are separated and concentrated into a single outlet (Collection Outlet), while the large cells and particles are concentrated into another outlet (Waste outlet). This allows increased purity of the samples to be analyzing. More specifically, when analyzing blood samples, or other fluids such as urine or sputum, the Acouwash™ removes the large red and white blood cells, leaving a solution rich in platelets, and in the case of an infection, microbes. The large blood cells would otherwise make the sample difficult to analyze, by cluttering the sample during flow microscopy, and physically blocking small microbes during imaging.

For the analysis of cell transduction success and apoptotic rates, the Acouwash™ allows for the filtration of particles accumulated from the use of Human serum as a growth media and the occurrence of cell lysis. This similarly removes clutter from the sample we are analyzing, allowing for clearer and more accurate results. The Acouwash™ is operated according to suggested parameters, as follows:

| Parameter | Setting Value | |
|---|---|---|
| Acoustic Wave Frequency | 2047 | Hz |
| Regulated Internal Pressure | 3 | atm |
| Sample Inlet Flow Rate | 50 | uL/s |
| Media Inlet Flow Rate | 100 | uL/s |
| Collection Outlet Flow Rate | 110 | uL/s |
| Waste Outlet Flow Rate | 40 | uL/s |

The separation media used is a 70 percent Ficole-Paque solution in PBS filtered by 0.2 μm PES filter during a vacuum filtration process. Biological samples, including bodily fluids such as blood, urine, and sputum, etc., are analyzed at a 10× dilution. After the appropriate dilution is made, each sample run processes at least 1 mL of sample to ensure the majority of the sample is properly separated. Prolonged processing minimizes entrance effects from the initiation of flow by allowing a large majority of the sample to be separated once the flow reaches a laminar state. Microbe sample concentrations were determined initially using the appropriate OD600 curve, and then confirmed by plating results. All plating experiments were done in triplicate.

Results in this embodiment are provided in Tables 1-3 below. In Table 1 the present inventors demonstrated with both E. coli and S. marcescens samples that nearly all of the microbes will be deposited in the collection outlet. In Table 2 it is demonstrated that this continues to be the case when microbes are in a murine blood environment, and in Table 3 the same results are demonstrated when microbes are in a human blood environment. From this data we can conclude that the separation step is being performed as intended, at an acceptable level of accuracy. We can expand upon the uses of acoustic separation, for example provided in this embodiment by the Acouwash™ to include processing of other fluids such as urine, and sputum. Additionally, the separation module, and in particular the exemplary Acouwash™ can be used to separate cell culture samples that may have debris from the media used, or from cell lysis occurring within the sample.

TABLE 1
Separation Efficiency of Microbes:

| Sample Identity | Percentage Recovered |
|---|---|
| E. coli Collection Outlet | 121.19 |
| E. coli Waste Outlet | 0.45 |
| S. marcescens Collection Outlet | 151.25 |
| S. marcescens Waste Outlet | 0.00 |

Table 1 above table displays the percentage of sample recovered based on the inlet concentration of microbe, for E. coli and S. marcescens samples. The collection outlet is the sample that will go on to be processed, and the waste is discarded. The apparent yield values which exceed 100 percent for the collection outlet are likely due to a reduction of the dilution factor during separation. All microbe inlet concentrations were diluted to 1000 CFU/mL.

TABLE 2
Separation Efficiency of Microbes in Murine Blood:

| Sample Identity | Percentage Recovered |
|---|---|
| Murine Blood and E. coli Collection Outlet | 111.86 |
| Murine Blood and E. coli Waste Outlet | 2.26 |

Table 2 above displays the percentage of sample recovered based on the inlet concentration of microbe, for E. coli in murine blood samples. The collection outlet is the sample that will go on to be processed, and the waste is discarded. The yields values which exceed 100 percent for the collection outlet are likely due to a reduction of the dilution factor during separation. All blood samples were run at a dilution factor of 10×, and all microbe inlet concentrations were diluted to 1000 CFU/mL

TABLE 3
Separation Efficiency of Microbes in Human Blood:

| Sample Identity | Percentage Recovered |
|---|---|
| Human Blood and E. coli Collection Outlet | 85.67 |
| Human Blood and E. coli Waste Outlet | 6.15 |

Table 3 above displays the percentage of sample recovered based on the inlet concentration of microbe, for E. coli in human blood samples. The collection outlet is the sample that will go on to be processed, and the waste is discarded. All blood samples were run at a dilution factor of 10×, and all microbe inlet concentrations were diluted to 1000 CFU/mL.

### Example 2. Microbial Analysis of Separated Biological Samples

Using the process set forth in the methods in Example 2 above, beginning with acoustic separation of the sample, followed by flow imaging microscopy, and ending with machine learning analysis, microbes can be isolated within an infected sample, and subsequently be identified by species. This process is a much more informative, accurate, and expedited form of diagnostic techniques currently available. This can be attributed to the methodical processing of the sample, and the high accuracy of species determination achieved by the invention's unique classifier.

Microbes were grown overnight in the appropriate media, and then pelleted before experimentation and re-suspended in filtered PBS (filtered with 2 μm filter using vacuum filtration). All culture samples were then diluted to a final dilution factor of 20× before image acquisition.

After the microbe samples are prepared, they are imaged using a the image capture modules, which as described above may include a high-throughput flow imaging instrument, such as the microfluidic device identified as the FlowCam Nano™, manufactured by Yokogawa Fluid Imaging Technologies. During this process, samples were pumped through a flowcell made of microscopy grade glass, 50 nm deep in the field of view and 500 nm wide. Images were continuously taken using high resolution oil microscopy, and FlowCam's™ particle detection algorithm segments the images so that a smaller image consisting only of an observed particle is output for every particle captured. These images were then compiled as tif files. The present inventors have adapted the FlowCam™ imaging acquisition parameters so that multiple instances of each particle are captured as it passes through the flow cell. This is achieved by minimizing the flow rate, maximizing the shutter speed and imaging rates, and increasing the flash duration to be nearly constant.

After the tif files are obtained, the images were processed by the machine learning modules, which is configured to apply and algorithm that has been developed specifically for this invention. In this preferred embodiment, the algorithm is a classifier which uses a cross-entropy loss function and a sliding window for classification determination, with a width of n=3, to make use of the replicate images. n=3 has been chosen because that is the most common number of replicates observed, but the parameter has been left tunable in order to leave room for future adaptations. The classifier functions using a two-step method, where initially ‘undesirable’ images are filtered out (such as platelets from blood samples), and then the ‘desirable’ images continue to a second classifier where microbe species identification occurs.

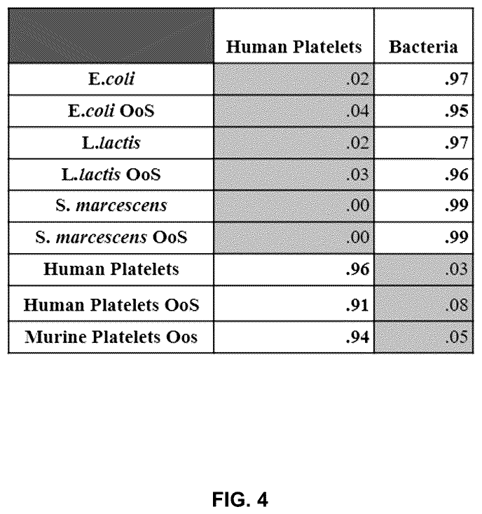

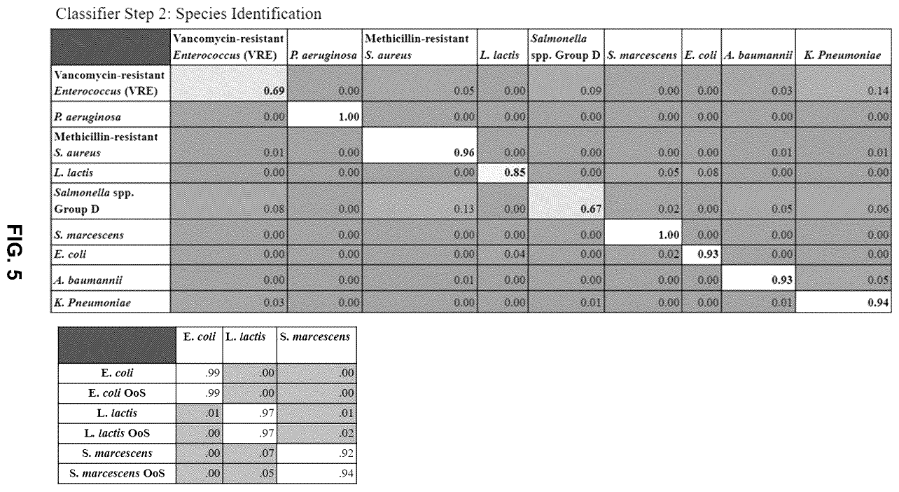

In FIG. 4 the results from the initial algorithm step, where microbes are differentiated from the host cells, is shown and demonstrates a high level of accuracy. All microbes have a level of accuracy pertaining to their ability to be separated from blood greater than 90 percent, with the exception of L. lactis with an accuracy of 89%, only in the first application Notably, 89% identification is sufficient to demonstrate that the algorithm can successfully differentiate L. lactis from blood. In the second table of the figure, three bacterial species are analyzed with out of sample (OoS) tests, meaning image sets on which the algorithm was not trained on. These all perform with high levels of accuracy, demonstrating the inventions capability to function using novel data, such as a human sample. Additionally, Murine platelets were included to demonstrate that this technique can be expanded to use with other species beside Humans. All of the images identified by the first classifier (5) as microbes are then passed into the second classifier (2) for species determination, as displayed in FIG. 5.

In FIG. 5 the species separation is demonstrated using a wide variety of species, including Bio-safety Level 1, Bio-safety Level 2, drug resistant, and non-drug resistant microbes. The accuracy of separation is very high, with all species classifying correctly a majority of the time, and over half of the species classifying with greater than 90 percent accuracy. These data demonstrate that the algorithm is proficient at accurately determining microbial species. In the second table out of sample (OoS) tests were again performed, and the classification is exceptional.

### Example 3. Detection of Murine and Human Blood Infections

Detection of microbial presence within murine or human blood samples is the natural continuation of the experiments and results presented in Example 2 described above. Detecting microbes within a blood sample allows for evidence that the machine learning algorithm can perform two important tasks: First, it can process and interpret data in which every part is foreign to the algorithm, and second, it can process a heterogeneous sample of host cells and microbes. The second task of processing a mixed sample further demonstrates the effectiveness of the inventive process.

Microbes were grown overnight in the appropriate media, and then the concentration was estimated using previously obtained OD600 growth curves. The samples were then diluted to a final dilution of 100,000 CFU/mL in Murine blood or 10,000 CFU/mL or 1,000 CFU/mL in Human blood. The final dilution factor of the Murine blood was 10× in filtered PBS (filtered with 0.2 μm filter using vacuum filtration), with an EDTA concentration of 2×. The final dilution factor of the Human blood was 30× in filtered PBS (filtered with 2 μm filter using vacuum filtration), with an EDTA concentration of 1×. The microbe concentrations were confirmed using plating results, performed in triplicate.

After dilution, blood samples are then processed using the Acouwash™, in order to obtain a microbe enriched sample with a higher degree of purity. The parameters for the operation of the Acouwash™ can be found in Example 1 above.

After the Murine and Human blood with added microbe samples were prepared, they were imaged image capture module, which in this embodiment included a FlowCam Nano™ as described above. During this process, samples were pumped through a flow cell made of microscopy grade glass, 50 nm deep in the field of view and 500 nm wide. Images were continuously taken using high resolution oil microscopy, and FlowCam's™ particle detection algorithm segmented the images so that a smaller image consisting only of an observed particle were output for every particle captured. These images were then compiled as tif files.

As noted above, the present inventors have adapted the FlowCam™ imaging acquisition parameters to increase the accuracy of detection and identification. The instrument's parameters are tuned so that multiple instances of each particle are captured as it passes through the flow cell. This is achieved by minimizing the flow rate, maximizing the shutter speed and imaging rates, and increasing the flash duration to be nearly constant.

After the tif files were obtained, the images are processed by the Machine learning module applying an algorithm of the invention which includes a classifier which uses a cross-entropy loss function and a sliding window for classification determination, with a width of n=3, to make use of the replicate images. n=3 has been chosen because that is the most common number of replicates observed, but the parameter has been left tunable in order to leave room for future adaptations. The classifier functions using a two-step method, where initially ‘undesirable’ images are filtered out (such as platelets from blood samples), and then the ‘desirable’ images continue to a second classifier where microbe species identification occurs.

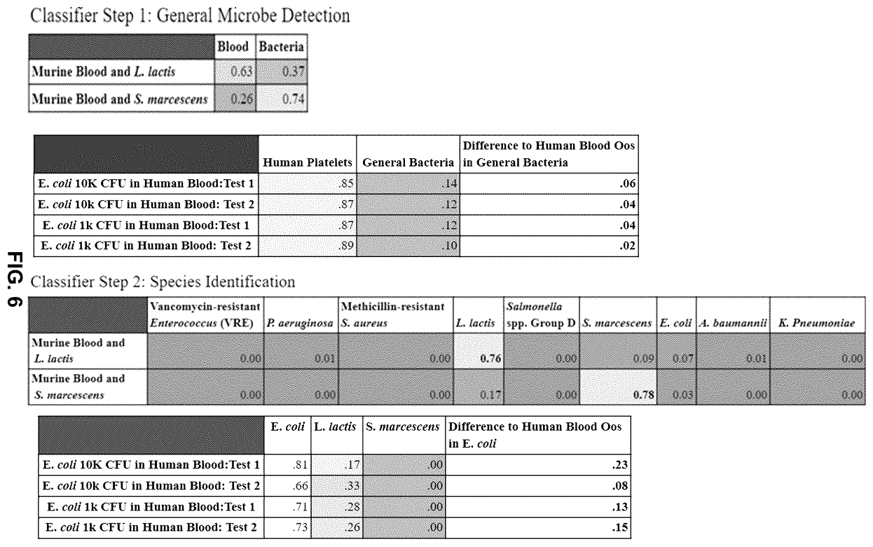

In FIG. 6 the results from the initial algorithm step, where microbes are differentiated from host cells, are shown and demonstrate that the algorithm is detecting microbes within the blood sample. The results are expected to be split because of the heterogeneity of the sample. In the case of the Human blood samples, an additional column is included representing the difference between the test samples percentage of bacteria detected, and what percentage is seen in a pure blood sample. The positive difference gives additional support that bacteria is being identified. All of the images identified as microbes in step one are then passed into the second step of the classifier for species determination.

In step two of the classifier the species identification of the pathogen is determined. The accuracy of identification is exceptional for the contaminated Murine blood samples, with all species classifying correctly a majority of the time. Specifically, for the L. lactis sample microbe images were positively identified 76 percent of the time, and for the S. marcescens sample images were positively identified 78 percent of the time. The identification of the E. coli within the human blood samples is also excellent. In the case of the Human blood samples, an additional column is included representing the difference between the test samples percentage of E. coli detected, and what percentage is seen in a pure blood sample. The positive difference gives additional support that E. coli is being identified correctly identified as the pathogen. These data demonstrate the algorithm of the invention is proficient at determining bacterial species within contaminated blood samples, including human and animal blood samples.

### Example 4. Detection of Urinary Tract Infections

In the following section the detection of microbial presence within urine samples is evaluated, demonstrating the application of this invention to urinary tract infection diagnosis. As described below, the proficiency of the invention in identifying infections within urine is demonstrated clearly, at the lowest microbial concentrations currently used to diagnose urinary tract infections. Using the lowest microbe concentration for diagnosis implies that infection will also be detected at higher microbe concentrations.

Microbes were grown overnight in the appropriate media, and then the concentration was estimated using previously obtained OD600 growth curves. The samples were then diluted to a final dilution of 1,000 CFU/mL in artificial urine. The final dilution factor of the urine was 10× in filtered PBS (filtered with 0.2 μm filter using vacuum filtration). The microbe concentrations were confirmed using plating results, performed in triplicate.

After dilution, urine samples can be then processed using the Acouwash™ instrument, in order to obtain a microbe enriched sample with a higher degree of purity, although in this application artificial urine was used and purification was unnecessary. The parameters for the operation of the Acouwash™ can be found in Example 1 above.

After the urine with added microbe samples were prepared, they were imaged using a FlowCam Nano™. During this process, samples were pumped through a flow cell made of microscopy grade glass, 50 nm deep in the field of view and 500 nm wide. Images were continuously taken using high resolution oil microscopy, and FlowCam's™ particle detection algorithm segments the images so that a smaller image consisting only of an observed particle is output for every particle captured. These images are then compiled as tif files.

As noted above, in this embodiment the present inventors have uniquely adapted the FlowCam™ imaging acquisition parameters to suit our project, and increase our accuracy of detection and identification. The instrument's parameters are tuned so that multiple instances of each particle are captured as it passes through the flow cell. This is achieved by minimizing the flow rate, maximizing the shutter speed and imaging rates, and increasing the flash duration to be nearly constant.

After the tif files were obtained, the images were processed by the machine learning module applying an algorithm of the invention. The algorithm in this embodiment includes a classifier which uses a cross-entropy loss function and a sliding window for classification determination, with a width of n=3, to make use of the replicate images. n=3 has been chosen because that is the most common number of replicates observed, but the parameter has been left tune-able in order to leave room for future adaptations. The classifier functions using a two-step method, where initially ‘undesirable’ images are filtered out (such as platelets from blood samples), and then the ‘desirable’ images continue to a second classifier where microbe species identification occurs.

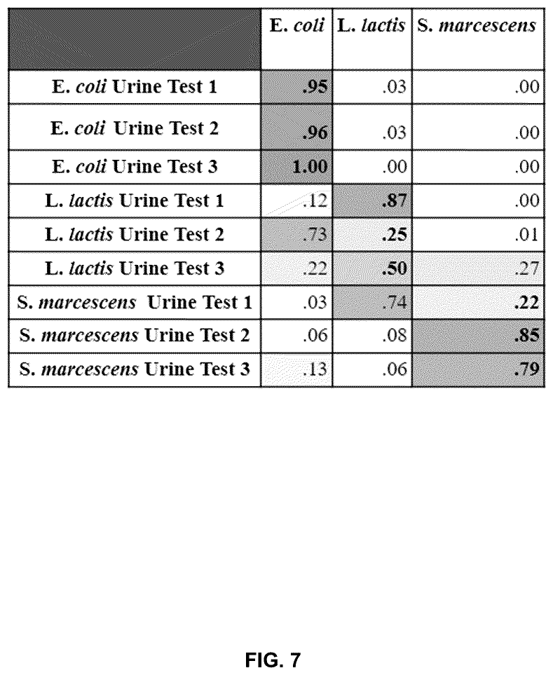

In FIG. 7 the results from the classifier for species identification of the pathogen are shown. The accuracy of identification is high for nearly all contaminated urine samples, with correct classifications for all E. coli tests, and 4 of the 6 test done for L. lactis and S. marcescens. These data are sufficient evidence to state that the algorithm is proficient at determining microbial species within contaminated urine samples, a majority of the time. Further, it is important to note the E. coli is the most common cause of urinary tract infections, whereas for L. lactis and S. marcescens urinary tract infections are rarely seen, if ever.

### Example 5. Determination of Pathogenic Microbe Drug Resistance and Toxicity

One aspect of this invention is to increase the accuracy of microbial infection diagnostic techniques, and therefore increase the likelihood of treatment success. An important aspect of this is determination of toxicity and drug resistant bacteria characteristics, after determination of the presence of an infection, and then subsequent identification of microbial species. Treatment options can be chosen based on the drug resistant properties of the microbe, and the microbe's degree of toxicity. Both of these can be identified using the systems and methods of the invention, as demonstrated below.

Microbes were grown overnight in the appropriate media, and then pelleted before experimentation and re-suspended in filtered PBS (filtered with 2 μm filter using vacuum filtration). All culture samples were then diluted to a final dilution factor of 20× before image acquisition.

After the microbe samples were prepared, they were imaged using a FlowCam Nano™ device. During this process, samples were pumped through a flow cell made of microscopy grade glass, 50 nm deep in the field of view and 500 nm wide. Images were continuously taken using high resolution oil microscopy, and FlowCam's™ particle detection algorithm segments the images so that a smaller image consisting only of an observed particle is output for every particle captured. These images were then compiled as tif files. Again, as noted above the present inventors have uniquely adapted the FlowCam™ imaging acquisition parameters to increase our accuracy of detection and identification. The instrument's parameters are tuned so that multiple instances of each particle are captured as it passes through the flow cell. This is achieved by minimizing the flow rate, maximizing the shutter speed and imaging rates, and increasing the flash duration to be nearly continuous.

After the tif files were obtained, the images were processed by the machine learning module of the invention. The algorithm is a classifier which uses a cross-entropy loss function and a sliding window for classification determination, with a width of n=3, to make use of the replicate images. n=3 has been chosen because that is the most common number of replicates observed, but the parameter has been left tune-able in order to leave room for future adaptations. The classifier functions using a two-step method, where initially ‘undesirable’ images are filtered out (such as platelets from blood samples), and then the ‘desirable’ images continue to a second classifier where microbe species identification occurs. For drug resistance determination, training data sets were grouped by drug resistances, instead of the typical grouping of subspecies or species. This allows for determination of microbe drug resistance properties, instead of determination of a specific sample type, which would not be as useful with evolving populations of microbes.

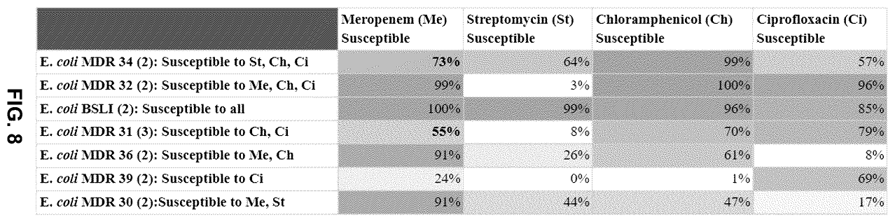

In FIG. 8 the bacteria have their drug resistance characteristics displayed, with a very high rate of accuracy when identifying any drug resistances. This classifier is novel in considerations of the other classifiers shown. In this application, each drug susceptibility and resistance pair was trained individually, and the results were then compiled. This allows for a 0 to 100% identification of susceptibility likelihood, for each individual case. Of the 28 cases shown in the table, only two misclassify, both in Meropenem susceptibility for the E. coli 34 and E. coli 31 cases. That being said the identifications are 73% and 55% respectively, the latter of which is very near the 50% threshold. With improved sampling and collection techniques, the results will improve to a higher level of accuracy. These results demonstrate that the invention is proficient at identifying any drug resistance characteristics of a microbe.

From these findings, the invention may be adapted to determine the degree of toxicity of microbe species, in addition to the drug resistances. This, in combination with the species and subspecies identifications, allows for an exact microbe to be determined during infection diagnostics.

### Example 6. Cell Therapy Viability Analysis

An additional expansion to the applications of this invention includes the analysis of transduction rates during the culture of CAR-T cells used in cell therapy for the treatment of cancer using immunotherapy.

For this application, the AcouWash™ is used to filter out the small particles present in cell culture from expected cell breakdown by cell lysis and the use of Human serum as a growth media. We then have a sample rich in T-cells, which is imaged using the FlowCam Nano™. The images are evaluated using a machine learning module of the invention, for which results are shown below. The AcouWash™ step in combination with the new customized FlowCam Nano™ settings allowing for multiple imaging of a single cell enhances both supervised (e.g., neural network-based logistic regression and triplet-loss based dimension reduction) and unsupervised machine learning algorithms (e.g., variational auto-encoders [VAEs] and generative adversarial networks [GANs]).

The creation of CAR-T cells is a complex process, in which T-cells are obtained from human blood, and activated after purification. After purification, lentiviral vector particles are used to transduce the activated T-cells, by delivering the CAR-transgene. The activated and transduced T-cells, herein called CAR-T cells, are then propagated, with the CAR-transgene propagating with them. In addition to the machine learning and imaging analysis that follows, all time points had flow cytometry performed in parallel, allowing for a comparison of results.

After the T-cell samples were prepared, they were imaged using a FlowCam Nano™ During this process, samples are pumped through a flow cell made of microscopy grade glass, 50 nm deep in the field of view and 500 nm wide. Images are continuously taken using high resolution oil microscopy, and FlowCam's™ particle detection algorithm segments the images so that a smaller image consisting only of an observed particle is output for every particle captured. These images were then compiled as tif files. As noted above, the inventors have adapted the FlowCam™ imaging acquisition parameters to increase the accuracy of detection and identification. The instrument's parameters are tuned so that multiple instances of each particle are captured as it passes through the flow cell. This is achieved by minimizing the flow rate, maximizing the shutter speed and imaging rates, and increasing the flash duration to be nearly continuous. The physical AcouWash™ method described in previous section can also be utilized to remove biological components that are smaller than the T-Cells in this embodiment.

After the tif files are obtained, the images are processed by machine learning module of the invention. One algorithm applied by the modules is a classifier which uses a cross-entropy loss function. The classifier functions using a filtering method, where initially ‘undesirable’ images are filtered out (in this case small debris particles and large cell aggregates that make it past the physical AcouWash™ sorting), and then the ‘desirable’ images continue to the classifier to be processed. For transduction rate determination, the training data sets are grouped by transduced samples, and non-transduced samples, at various time points. An example of this application in shown in FIG. 9, using supervised learning. For unsupervised learning, transduced and/or non-transduced cells at various time points can be used to estimate low-dimensional representations of the images of interest via techniques involving VAEs and GANs. Unsupervised representations can be visualized for exploratory data analysis (EDA) or quality control (QC) applications using additional post-processing steps such as t-distributed stochastic neighbor embedding (t-SNE) or Uniform Manifold Approximation and Projection (UMAP) techniques; Example 10 and 11 illustrate some examples of VAEs and UMAP with different HTIs.

### Example 7. Bacterial Transduction Rate Analysis

An additional expansion to the applications of this invention includes the analysis of transduction rates during the transfection of bacterial cells used in industry and research for the production of proteins and other biological molecules.

For this application, the AcouWash™ is used to filter out the small particles present in cell culture from expected cell breakdown by cell. We then have a sample rich in bacterial cell, which is imaged using the FlowCam Nano™ The images are evaluated using a machine learning module of the invention, for which results are shown in FIG. 10. The AcouWash™ step in combination with the new customized FlowCam Nano™ settings allowing for multiple imaging of a single cell enhances both supervised (e.g., neural network-based logistic regression and triplet-loss based dimension reduction) and unsupervised machine learning algorithms (e.g., variational auto-encoders [VAEs] and generative adversarial networks [GANs]).

During this process, samples are pumped through a flow cell made of microscopy grade glass, 50 nm deep in the field of view and 500 nm wide. Images are continuously taken using high resolution oil microscopy, and FlowCam's™ particle detection algorithm segments the images so that a smaller image consisting only of an observed particle is output for every particle captured. These images were then compiled as tif files. As noted above, the inventors have adapted the FlowCam™ imaging acquisition parameters to increase the accuracy of detection and identification. The instrument's parameters are tuned so that multiple instances of each particle are captured as it passes through the flow cell. This is achieved by minimizing the flow rate, maximizing the shutter speed and imaging rates, and increasing the flash duration to be nearly continuous. An example of this application is shown in FIG. 10. Transduced and Non-transduced samples are analyzed, of the bacteria B. subtilis. Out of sample (OoS) tests were performed to test the validity of the results. The correct classification occurred 75% of the time, with only the Transduced OoS test misclassifying. With continued and prolific testing in the future this will be able to be improved and provide higher levels of accuracy. The results currently show that our algorithm is proficient at identifying transduced vs. non transduced samples, expanded to included various bacterial species.

### Example 8. Additional Diagnostic Applications

Using the information obtained from the algorithm results, the present inventors can determine the concentration of bacteria within a sample, and subsequently determine the severity of infection. This allows appropriate measures to be taken regarding patient care, and the rigor of treatment needed. The calculation of CFU/mL can be performed using the number of microbes identified within a specified sample volume, the average number of repeat image instances expected, and the volumetric percentage of fluid captured during imaging.

As mentioned in the initial description, this invention can be used as a diagnostic technique for any microbial disease where the microbe is present in a bodily fluid, or where the microbe can be obtained and cultured in media, for example when a scrape or swab is performed to diagnose a skin infection. Some examples of biological samples this invention would be able to process and determine the presence of an infection for include blood, urine, sputum, cerebrospinal fluid, amniotic fluid, joint fluid, and mucus. Experimental examples of fluid infection evaluation are shown in Examples 3 and 4.

The benefits of real-time monitoring are numerous. This invention is uniquely suited to perform real-time monitoring of patient condition for two main reasons. First, this method of diagnosis is much faster than current diagnostic methods. for example, what would normally be a 3 to 5 day process is reduced to an hour. Second, because of the time reduction, and the lack of materials needed for the diagnostic procedure, the monitoring of patient condition would be inexpensive both monetarily and in regards to the time consumption of medical personnel. One example for how real-time monitoring can be used is to evaluate treatment effectiveness. For example, the monitoring of a patient's microbe concentration in their blood while undergoing treatment could provide insight into the degree of treatment success, and allow a physician to alter treatment as needed for optimum patient recovery.

In addition to analysis of treatment effectiveness, this invention could be used for disease prevention. In one embodiment, this can be demonstrated when analyzing the risks of long-term catheter use. In hospitalized patients, especially elderly patients, sepsis is serious disease that often develops from undetected urinary tract infections (caused by long-term catheter use) that spread from the urinary track to the blood or other organ systems. Monitoring the urine and/or blood for infection could prevent a life-threatening disease from ever occurring.

### Example 9. Using Unsupervised Embedding Representations to Characterize CAR-T Cell Morphologies Encoded in Images Obtained with Dynamic Imaging

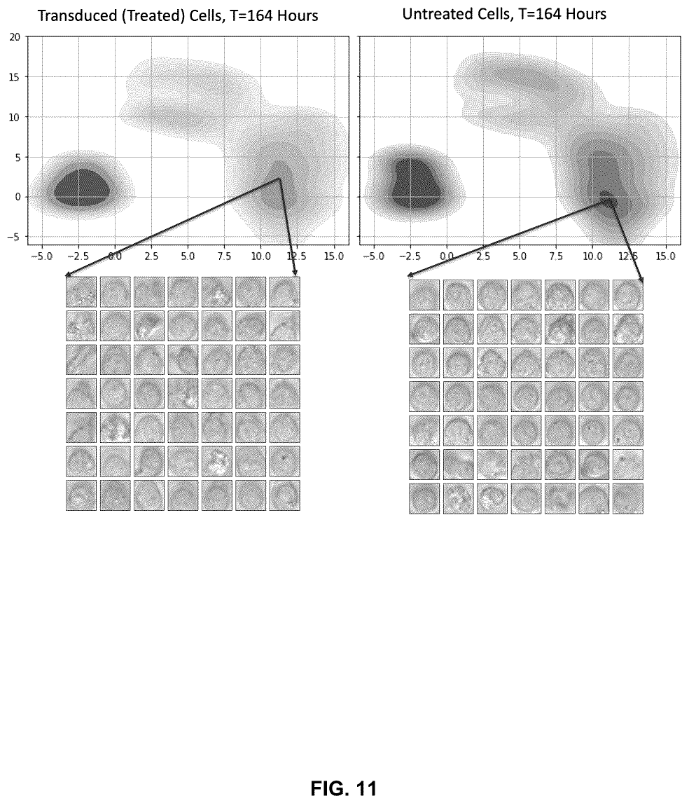

This example shows an alternative analysis to that in Example 6. FIG. 11 illustrates how a neural network trained in an unsupervised fashion with a mean square error reconstruction loss using FlowCam Nano images of transduced and non-transduced CAR-T cells can help in exploring the different morphologies present in two different heterogeneous cell populations. No acoustic separation or classification step was applied in the analysis (separation was achieved by estimated particle size filtering). The latent space of a variational autoencoder was reduced to two dimensions by Uniform Manifold Approximation and Projection (UMAP) in the top panels; the top panels display the probability density function obtained by kernel density estimate (kde) of the UMAP representation of multiple particles imaged from two different cell conditions. The left panel displays transduced CAR-T cells and the right panel displays non-transduced (untreated) CAR-T cells. Each region in the top panel corresponds to different particle morphologies. To illustrate, two separate regions in the UMAP kde are shown; the corresponding source FlowCam Nano images for the ˜50 points nearest the arrow are shown in the bottom panels. Over time, the population density shifts for both the transduced and untreated cell populations and the dynamics depend on whether or not transduction was applied. These changing morphologies for different conditions can be quantitatively monitored and characterized through the embeddings output by the machine learning module.

### Example 10. Using Unsupervised Embedding Representations to Characterize Protein Aggregate Morphologies Encoded in Images Obtained by Using Multiple Signal Modalities

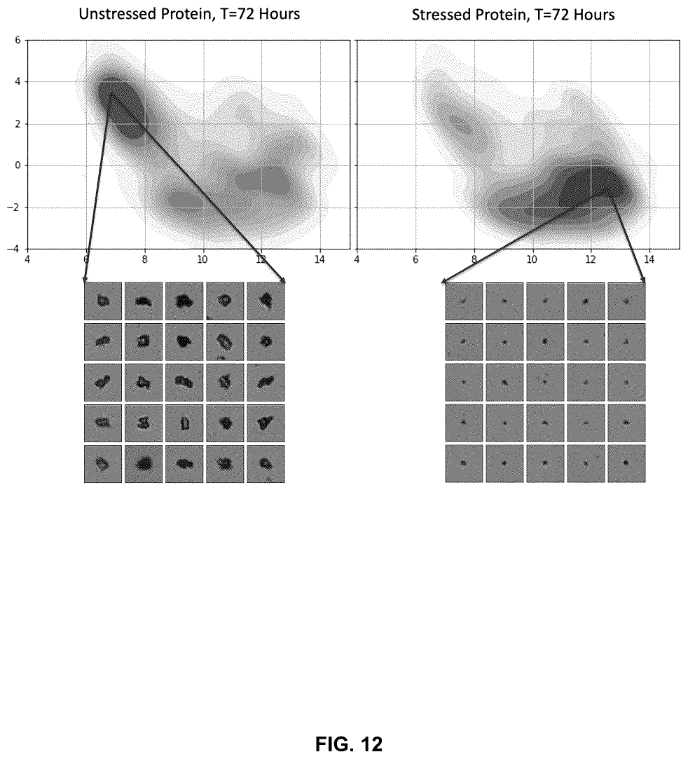

An additional expansion to the applications of this invention includes the analysis of the morphology of protein aggregates extracted from antibody formulations. FIG. 12 illustrates how a neural network trained in an unsupervised fashion with a mean square error reconstruction loss using two separate image channels of a HORIZON Backgrounded Membrane Imaging (BMI) system can help in exploring the different morphologies present in two nominally identical antibody formulations experiencing different stress conditions. Brightfield and darkfield images of particles were used to train and then analyze mechanically agitated and unstressed antibody formulations. No acoustic separation or classification step was applied in the analysis (separation was achieved by estimated particle size filtering). The latent space of a variational autoencoder (with two separate image channel inputs) was reduced to two dimensions by Uniform Manifold Approximation and Projection (UMAP) in the top panels; the top panels display the probability density function obtained by kernel density estimate (kde) of the UMAP representation of multiple particles imaged from two different stress conditions. The left panel displays particles observed in unstressed antibody solutions and the right panel displays particles from mechanically agitated (via plate shaker) antibody solutions. Each region in the top panel corresponds to different particle morphologies. To illustrate, two separate regions in the UMAP kde are shown; the corresponding source brightfield BMI images for the ˜50 points nearest the arrow are shown in the bottom panels. The population density depends on the stress history experienced by the antibody formulation. These distinct morphologies for different conditions can be quantitatively monitored and characterized through the embeddings output by the machine learning module. In this application, the variational autoencoder fused brightfield and darkfield measurements. However, other images, such as those from a fluorescence image channel or a raman spectra could be used in addition or in place of inputs shown in this example. The embeddings and latent space of the variational autoencoder can also be used to detect for the presence of unexpected contaminants (such as glass, silicone oil, fibers, etc.) in the antibody formulation. Other quality control applications include using the measured embeddings to check for the consistency of particle morphology and distribution in products nominally manufactured identically.

1. A system for analyzing a biological sample comprising:
a biological sample containing a quantity of microparticles;
an image capture module configured to capture a plurality of digital image signals of said microparticles present in said biological sample;
a machine learning module configured to process the digital image signals from said image capture module further comprising:
a digital filter to differentiate the images of microparticles of interest from images of microparticles in said biological sample; and
a convolutional neural network configured to further identify said microparticles of interest.

2. The system of claim 1, further comprising a separation module configured to separate the microparticles in said biological sample into a collection outlet stream containing predominantly microparticles of interest, and a waste stream containing predominately other particles found in said biological sample.

3. The system of claim 2, wherein said image capture module is further configured to capture a plurality of digital image signals of said microparticles present in said collection outlet of said biological sample.

4. The system of claim 1, wherein said digital filter is further configured to differentiate images of microparticles of interest from images of the microparticles in said collection outlet of said biological sample.

5-8. (canceled)

9. The system of claim 1, wherein said wherein the biological sample comprises a biological sample selected from the group consisting of: sputum, oral fluid, amniotic fluid, blood, a blood fraction, bone marrow, a biopsy samples, urine, semen, stool, vaginal fluid, peritoneal fluid, pleural fluid, tissue explant, mucous, lymph fluid, organ culture, cell culture, a fraction or derivative thereof or isolated therefrom, or a static or flowing liquid suspension.

10. (canceled)

11. The system of claim 1, wherein said microparticles in said biological sample are selected from: microbial microparticles, non-microbial microparticles, cells, or pathogenic microbes.

12-15. (canceled)

16. The system of claim 1, wherein said image capture module comprises a high-throughput imaging instrument capable of imaging flowing or static suspensions of microparticles, or a high-throughput microfluidic imaging instrument capable of imaging flowing or static liquid suspensions.

17-20. (canceled)

21. The system of claim 1, wherein said digital filter comprises a convolutional neural network further comprising a machine learning-based automated classifier configured to determine if the microparticles are a microbe of interest, or a subject-derived cell, and/or wherein said digital filter comprises a convolutional neural network further comprising a machine learning-based embedding scheme configured to determine if the cell culture components comprising the microparticles are microbes of interest, or a subject-derived cells.

22. The system of claim 21, wherein said convolutional neural network comprises a machine learning-based automated classifier configured to identify the microbe of interest by genus, species, phenotypic characteristic, genotypic characteristic, or one or more antibiotic resistance characteristics.

23-27. (canceled)

28. The system of claim 1, wherein said machine learning module further comprises a fusion module adapted to combine signal modalities.

29-30. (canceled)

31. The system of claim 1, wherein said machine learning module comprises a machine learning module configured to extract one or more features of said microparticles of interest by machine learning including supervised learning, or unsupervised learning.

32-33. (canceled)

34. A system for characterizing changes in cell populations:
a biological sample containing a quantity of a cell culture further containing a quantity of engineered cells;
an image capture module configured to capture a plurality of digital image signals of the engineered cells present in said biological sample;
a machine learning module configured to process the digital image signals from said image capture module further comprising a convolutional neural network configured to extract a feature of interest from said images;
one or more additional biological samples containing a quantity of a cell culture containing a quantity of engineered cells applied to the system above and compared to said preceding biological sample, wherein said comparison identifies a characteristic change in said engineered cells between the samples.

35. The system of claim 34, further comprising a separation module configured to separate said engineered cells in said biological sample into a collection outlet of said biological sample.

36-40. (canceled)

41. The system of claim 34, wherein said feature of interest comprises a feature of interest associated with transduced cells, or non-transduced cells.

42. The system of claim 41, wherein said transduced cells comprise T cells transduced to form CAR-T cells.

43-48. (canceled)

49. The system of claim 34, wherein said digital filter comprises a machine learning-based automated classifier configured to determine if the cell culture components comprise T cells transduced to form CAR-T cells, or non-transduced T-cells.

50. The system of claim 34, wherein said digital filter comprises a machine learning-based embedding scheme configured to determine if the cell culture components comprise transduced CAR-T cells, or non-transduced T-cell.

51-54. (canceled)

55. The system of claim 34, wherein said machine learning module further comprises a fusion module adapted to combine signal modalities, or to fuse embeddings in signals from two or more modalities.

56-57. (canceled)

58. The system of claim 34, wherein said machine learning module comprises a machine learning module configured to extract one or more features of interest by supervised learning or unsupervised learning.

59. A system for characterizing changes in pharmaceutical sample populations:
a pharmaceutical sample containing a quantity of microparticles;
an image capture module configured to capture a plurality of digital image signals of the microparticles present in said pharmaceutical sample;
a machine learning module configured to process the digital image signals from said image capture module further comprising a convolutional neural network configured to extract a feature of interest from said images; and
one or more additional pharmaceutical samples containing a quantity of microparticles applied to the system above and compared to said preceding pharmaceutical sample, wherein said comparison identifies a characteristic change in between the samples.

60-79. (canceled)
